(19) United States
(12) Patent Application Publication
(10) Pub. No.: US 2025/0296278 A1
(43) Pub. Date: Sep. 25, 2025

(54) CHAOTIC PRINTING FOR THE PRODUCTION OF NON-FILAMENTOUS ARCHITECTURES

(71) Applicant: OHIO STATE INNOVATION FOUNDATION, Columbus, OH (US)

(72) Inventor: David DEAN, Columbus, OH (US)

(21) Appl. No.: 18/861,795

(22) PCT Filed: May 1, 2023
(86) PCT No.: PCT/US23/20626

§ 371 Date: Oct. 30, 2024

Related U.S. Application Data
(60) Provisional application No. 63/337,094, filed on Apr. 30, 2022.

(51) Int. Cl. B29C 64/118; A61L 27/36; A61L 27/54; B29C 64/336; B29K 1/00; B33Y 10/00; B33Y 40/20; B33Y 80/00

(52) U.S. Cl. CPC B29C 64/118; A61L 27/3687; A61L 27/3691; A61L 27/54; B29C 64/336; B33Y 10/00; B33Y 40/20; B33Y 80/00; B29K 2001/08; B29K 2995/0056

(57) ABSTRACT

Disclosed are methods for preparing non-filamentous scaffolds (e.g., sheets) for cell or tissue culture. These methods can comprise providing at least a first printing composition (e.g., a bioink) and a second printing composition (e.g., a bioink or a fugitive ink); chaotic printing the first printing composition and the second printing composition to generate a microstructured precursor comprising a plurality of lamellar structures formed from the first printing composition and the second printing composition; extruding the microstructured precursor through a nozzle (e.g., a fan-shaped nozzle, a curved fan-shaped nozzle, or an annular nozzle) to produce a non-filamentous microstructured precursor; and curing the non-filamentous microstructured precursor to provide the non-filamentous scaffold for cell or tissue culture.

### CROSS-REFERENCE TO RELATED APPLICATIONS

This application claims benefit of U.S. Provisional Application No. 63/337,094, Apr. 30, 2022, which is hereby incorporated herein by reference in its entirety.

### BACKGROUND

Vascularization is a key challenge to tissue engineering. While many developing strategies have incorporated capillary beds into tissue engineered constructs, they are prevented from becoming clinically viable by their inability to establish long-term blood perfusion from native arteries to all regions of the construct and back to veins. As a result, autografts remain the clinical gold standard for tissue repair despite significant advantages that tissue engineering could alleviate. Perfusion must occur quickly upon implantation to allow engineered tissues to persist by providing oxygen and nutrient delivery and metabolic byproduct removal. Moreover, there cannot be distances in the construct greater than 200 microns (i.e., the diffusion limit of oxygen and nutrients) without direct capillary contact, a requirement increasingly difficult to meet with larger, more physiologically-relevant tissue and organ constructs.

The cardiovascular system accomplishes the feat of complete perfusion to natural tissues through a hierarchical organization of arteries and veins branching into microvasculature (arterioles and venules) that provides homogenous distribution of blood to capillary beds. It is recognized in the field that recreating this native network organization is crucial to perfusing large-scale engineered tissue and organ grafts, with an emphasis on achieving the transition from artery/vein to capillary-scale perfusion that microvasculature provides. With only a few select tissues in the body being avascular (i.e., not supplied directly by blood vessels), such as the lens and cornea of the eye, epithelial layer of skin, and cartilage, the problem of perfusion limits efforts to reach clinical viability in essentially every other area of tissue engineering. However, as yet, strategies for replicating the hierarchically branching networks of microvasculature that distribute homogeneous blood supply from arteries to capillary beds in natural tissues are lacking.

### SUMMARY

Provided herein are methods for preparing scaffolds for cell or tissue culture. The scaffolds can be non-filamentous. For example, the scaffolds can be in the form of a sheet, a curved sheet, a hollow tube, or a multilayer sheet. These methods can comprise providing at least a first printing composition and a second printing composition; chaotic printing the first printing composition and the second printing composition to generate a microstructured precursor comprising a plurality of lamellar structures formed from the first printing composition and the second printing composition; extruding the microstructured precursor through a nozzle to produce a non-filamentous microstructured precursor; and curing the non-filamentous microstructured precursor to provide the non-filamentous scaffold for cell or tissue culture.

In some embodiments, the nozzle exhibits a substantially non-circular cross-section. In some embodiments, the non-filamentous microstructured precursor and the non-filamentous scaffold exhibit a substantially non-circular cross-section perpendicular to an axis along which extrusion occurs.

In some examples, the nozzle can comprise a fan-shaped nozzle. In these embodiments, the non-filamentous microstructured precursor and the non-filamentous scaffold for cell or tissue culture can comprise a sheet or multilayer sheet. In some embodiments, the sheet can have a width and a height, and the width of the sheet can be at least five times (or at least ten times) the height of the sheet.

In other examples, the nozzle can comprise a curved fan-shaped nozzle or annular nozzle. In these embodiments, the non-filamentous microstructured precursor and the non-filamentous scaffold for cell or tissue culture can comprise a curved sheet or hollow tube.

In some embodiments, the method can further comprise using a multiplexer to select various chaotically printed microstructured precursors that are co-extruded to produce the non-filamentous microstructured precursor.

In some embodiments, the first printing composition comprises a bioink composition.

In some embodiments, the second printing composition also comprises a bioink composition. In some of these examples, the first printing composition and the second printing composition are of different composition.

In other embodiments, the second printing composition comprises a fugitive ink composition. In these embodiments, the methods can further comprise removing the fugitive ink composition from the non-filamentous scaffold following curing.

In some embodiments, the method can further comprise bioprinting, electrospinning, and/or melt electrowriting a third printing composition onto or into the non-filamentous scaffold. In some examples, the third printing composition can comprise a bioink composition. In some of these examples, the third printing composition can be of a different composition than the first printing composition and the second printing composition.

In some embodiments, each of the bioink compositions individually comprises a polymer. In some examples, the polymer can comprise a hydrogel-forming agent. In some examples, the polymer can comprise a polysaccharide, such as alginate, hyaluronic acid, agarose, or any combination thereof. In some examples, the polymer can comprise a protein or peptide, such as gelatin, collagen, or any combination thereof. In some examples, the polymer can comprise a synthetic polymer, such as a polyester (e.g., poly(propylene fumarate) (PPF), polycaprolactone, poly(lactic-co-glycolic acid), polylactic acid, polyglycolic acid, or any combination thereof). In some examples, the polymer is crosslinkable. In some examples, the polymer can be present in an amount of from 0.5% to 20% by weight, based on the total weight of the bioink composition. In some embodiments, the bioink composition can further comprise cells (e.g., pluripotent stem cells, multipotent stem cells, progenitor cells, terminally differentiated cells, endothelial cells, endothelial progenitor cells, immortalized cell lines, primary cells, or any combination thereof).

In some embodiments, the method can further comprise dispersing a population of cells in the bioink composition (e.g., the first printing composition and/or the second printing composition, when the second printing composition comprises a bioink) prior to the chaotic printing. In some embodiments, the method further comprises seeding the non-filamentous scaffold with a population of cells. In these embodiments, the cells can comprise, for example, pluripotent stem cells, multipotent stem cells, progenitor cells, terminally differentiated cells, endothelial cells, endothelial progenitor cells, immortalized cell lines, primary cells, or any combination thereof.

In some embodiments, the fugitive ink composition, when present, comprises a polymer. In some examples, the polymer can comprise a poly(alkylene oxide) block copolymer, such as a polyoxyethylene-polyoxypropylene (PEO-PPO) block copolymers (e.g., a poloxamer). In some examples, the polymer can comprise hydroxyethyl cellulose (HEC). In some examples, the polymer can be present in an amount of from 0.5% to 20% by weight, based on the total weight of the fugitive ink composition.

In some embodiments, chaotic printing of the first printing composition and the second printing composition can comprise inducing laminar flow of the first printing composition and the second printing composition through a mixer that chaotically mixes the first printing composition and the second printing composition to form lamellar interfaces between the first printing composition and the second printing composition.

In some embodiments, chaotic printing of the first printing composition and the second printing composition can comprise coextruding the first printing composition and the second printing composition through a mixer that chaotically mixes the first printing composition and the second printing composition to form lamellar interfaces between the first printing composition and the second printing composition.

In some embodiments, the mixer can comprise a static mixer, such as a Kenics static mixer.

In some embodiments, chaotic printing of the first printing composition and the second printing composition can comprise coextruding the first printing composition and the second printing composition with a crosslinking agent. In some examples, the first printing composition comprises an alginate and the crosslinking agent comprises a divalent cation. In certain examples, the crosslinking agent comprises a calcium salt such as calcium chloride.

In some embodiments, the non-filamentous scaffold exhibits an average striation thickness of from 10 nm to 200 μm.

In some embodiments, the non-filamentous scaffold exhibits a surface-area-to-volume (SAV) of from 400 m⁻¹ to 5000 m⁻¹.

In some embodiments, the non-filamentous scaffold exhibits a surface density of at least 0.05 m² cm⁻³.

Also provided are scaffolds for cell or tissue culture, including microvascular appendage sheets, prepared by the methods described herein.

### DESCRIPTION OF DRAWINGS

FIGS. 1A-1C illustrate embryological development and wound healing. The function of many organs depends on membranes having a thickness of a single cell. Gastrulation is the indentation and apposition of two membranes, two sheets of a single layer thick, which results in the induction (i.e., production) of mesoderm. The single cell thick layers that form the nephron in the kidney or the alveolus in the lung derive from adjacent cell layer's secreting cell-cell signaling molecules, a process termed “induction.”

FIG. 2 illustrates and compares standard nozzle based bioprinting (top left) and chaotic printing (bottom left). In standard bioprinting filaments (1-5 mm diameter) only contact at their extremes making it impossible to produce thin membranes, sheets, of two different types of cells in close adjacency. Chaotic printing can use chaotic flows or shear, as is shown here, to produce layers as thin as a single or a few cells (e.g., 50 μm thick) in complete adjacency with layers of different types of cells. This allows those cells to directly interact and bring about organ-level function. Note that when two types of cells are included in a standard bioprinter they distribute homogeneously rather than into layers as is in chaotic printing.

FIG. 3 illustrates chaotic flows: paint is poured down the center rod (attached to belt and motor upper right) into a larger cylinder. The portion of the center rod sitting in the lower cylinder has a slit from which it releases paint into the lower cylinder. Layers are short and mix slowly when both cylinders rotate in the same direction (concentric circles of color at upper right). However, if either cylinder turns in the opposite direction, chaotic flows produce very long lamina. Similar laminar flows can be produced in a Kenics Static Mixer. The key to chaotic printing is to shape and preserve cell-bearing lamina. (FIGS. 1A-1C and 2).

FIGS. 4A-4D illustrate aspects of the Kenics Static Mixer (KSM). As shown in FIG. 4A, each KSM element (blade) rotates its bioink input 90 degrees where it then either meets another blade (FIG. 4B) which splits (doubles) its content into new layers, or it is extruded. As shown in FIG. 4C, fugitive ink can provide channels for cell culture media flow in a bioreactor or allow for later seeding of cells, such as endothelial cells. As shown in FIG. 4D, if rheological conditions are correct, the extrusion nozzle can be extremely long without damaging the cells or mixing the layers. This fact also makes the technologies envisioned in FIG. 6 possible.

FIG. 5 illustrates microvasculature formation: Top Left: The adult deep femoral artery (DFA or profunda femoris artery; 2.5-7.5 mm diameter) 22 supplies thigh muscles and femur. First inset: DFA arterioles (100 μm<diameter) pierce periosteum, and run vertically outside cortex, branches enter Volkmann's canals. Second inset: capillary beds form in osteons. Right column: microvascular patterns of different tissues. Bottom row: Timing of formation vascular branches connecting arteries with dense microvasculature made up of arterioles and capillary beds through looping, anastomoses, parsing of arterioles and sprouting of capillaries.

FIG. 6 illustrates the multimodality of chaotic printing. Sheet chaotic printing can be combined with standard bioprinting, electrospinning, and melt electrowriting. In next generation chaotic printing, flat sheet printing can be extended to produce curving sheets, concentric tubular layers, hollow walls, and solid-layered tissues (functional portions) within organs.

FIG. 7 illustrates the aims of example 1: Aim 1: Fabrication and cell expansion in filaments. Aim 2: Differentiation in filaments or sheets. Aim 3: Rat femur mode.

FIG. 8A shows an optical micrograph cross-section of a construct of C2C12 cells (mouse myoblast) chaotically printed in an alginate/GelMA hydrogel using a 4-layer KSM printhead (scale bars [sb]: 500 μm and 50 μm, respectively).

FIG. 8B shows an SEM micrograph cross-section of a construct of C2C12 cells (mouse myoblast) chaotically printed in an alginate/GelMA hydrogel using a 4-layer KSM printhead (scale bars [sb]: 500 μm and 50 μm, respectively).

FIG. 8C shows a longitudinal view of a chaotically-printed construct; high cell viability. Inset shows a cross section (sb: 500 μm).

FIG. 8D shows that C2C12 cells attach, proliferate, and migrate within chaotically-printed layers containing RGD (day 13). (sb: 200 μm).

FIG. 8E shows an optical microscopy view of a segment of a filament containing C2C12 cells (day 18) showing maintenance of position in originally printed layers (sb: 500 μm).

FIG. 8F shows staining for F-actin and DAPI for nuclei shows cell spreading and the formation of interacting cell clusters (cell nuclei are blue; Actin filaments are red; sb: 200 μm).

FIGS. 9A-9E show the chaotic printing of HUVEC and MCF7 (immortalized breast cancer) using a first-generation chaotic printing device based on the mini-JB: FIG. 9A shows sheets of HUVECs expressing green fluorescent protein (HUVEC-GFP) were chaotically printed in a GelMA. FIG. 9B shows a different focal plane of the same region.

FIG. 9C shows computer re-planarized images showing similar layer features (Scale bars [sb: 500 μm); FIGS. 9D and 9E show HUVEC-GFP chaotically printed in GelMA containing VEGF. HUVECs first spread along a plane defined by the chaotically-printed layer's surface and, subsequently, migrate along those surfaces at 96 hours (sb: 100 μm).

FIG. 10 shows the effect of fugitive channels in chaotically-printed filaments. PrestoBlue-tracked BM-hMSC proliferation in GelMA layers with and without HEC fugitive layers.

FIG. 11 illustrates intercellular network formation: hMSC/HUVEC spheroids in fibrin/poly(propylene fumarate) composite scaffolds via confocal (E-H) microscopy. (Scale bar 200 μm in E-G and 50 μm in H. Cells sprouted throughout the fibrin scaffold. Immunofluorescent staining was performed for the endothelial cell marker CD31 (TRITC, red) and the hMSC marker α-SMA (FITC, green). Z-stack images were taken using confocal laser-scanning microscopy and two-dimensional projections are shown. CD31-positive vessel-like structures are seen after 1 week, 2 weeks (E, H), and 3 weeks (F-G) of culture. (G) CD31/α-SMA merged image shows co-localization of hMSCs with HUVEC sprouts that extend to the edge of the scaffold. (H) CD31/α-SMA merged image shows hMSC/HUVEC interactions and visible lumen-like structures after 2 weeks of culture. (I) CD31-positive stained area was quantified by confocal microscopy. Microvascular network area increased with culture time.

FIGS. 12A-12B show the macroscopic and microscopic lectin fluorescence imaging of neomicrovascular perfusion. FIG. 12A shows an analysis of the explanted fibrin scaffolds to quantify the total florescence radiant efficiency after lectin tail vein injection. FIG. 12B shows a microscopic fluorescence analysis of the positive lectin staining of the vascular areas (red) along with the cell nuclei indicated by DAPI staining (blue) at the fibrin area inside the scaffold shows the spreading and increase of vascular networks upon moving sequentially through the no pre-culture (NP), 1 week pre-culture (1P), 2-week pre-culture (2P) and 3-week pre-culture (3P) groups. Here, the control (C) group does not show any positive staining for lectin demonstrating the absence of vessels inside the scaffold. (Scale bar for all images shown in FIG. 12B is 100 μm.).

FIGS. 13A-13D shows the characterization of microvasculature in acellular lung matrix. FIG. 13A shows a 3D micro-CT of the acellular matrix airway compartment. Large airways are in green. FIG. 13B shows micro-CT angiography of vascular compartment, thresholded to visualize only macro and micro-vasculature. (The scale bar in FIGS. 13A and 13B is 4 μm.) FIG. 13C shows micro-CT angiography of smaller vessels in acellular lung. (The scale bar in FIG. 13C is 500 mm.) FIG. 13D is an immunoblot for MHC-1, MHCII and b-actin in native (Nat) and decellularized (Dec) lungs, showing removal of cellular proteins.

FIG. 14A illustrates the local flap” method applied via (1) “rotation” and (2) “tunneling” techniques.

FIG. 14B illustrates the “Free flap” method.

FIG. 15A shows the approximate scale of blood vessels in the body. Veins and venules roughly follow the same dimensions as arteries and arterioles, respectively.

FIG. 15B illustrates the process of “Vasculogenesis” in endothelial cells.

FIG. 15C illustrates the process of “Angiogenesis” in endothelial cells.

FIG. 16A shows a chaotic printing system design for producing filaments.

FIGS. 16B and 16C show how individual KSM elements in succession bifurcates the flow of each ink into adjacent layers within a single filament.

FIG. 16D shows a syringe pump chaotically printing into CaCl₂) for gel crosslinking.

FIG. 16E show a fiber cross-section showing alternating layers of the initial two inks.

FIG. 17 illustrates an example MAS System Design. (A) First, two bioinks are mixed into varying layer numbers in separate printheads, then a multiplexer determines which mixture is sent to the nozzle to control layer number and thickness in the resulting sheet. (B) The nozzle fans the bioink mixture into a wide sheet. (C) MAS cross-section. (D) During the in vitro pre-vascularization stage, supporting cells such as pericytes can surround newly formed endothelial cell lumens to provide chemical and mechanical cues. (E) After pre-vascularization, the MAS can be sutured to native vessels and wrapped around tissue to provide new blood supply.

FIG. 18 illustrates a multiplexer design concept. The circular device rotates to select between multiple flow inputs.

FIG. 19 illustrates how incorporating green fluorescent particles into one of the two hydrogel inputs is a simple way to quickly observe alternating layers in chaotically printed constructs

FIGS. 20A-20E illustrate an example of how confocal microscopy can be used to observe lumen formation and reconstruct 3D images of engineered microvasculature.

FIG. 21 illustrates three example inner layer designs: (V1) alternating, hierarchically branching layers of solid HUVEC-laden hydrogel and solid PC-laden hydrogel, (V2) alternating, hierarchically branching layers of solid HUVEC and hPC-laden hydrogel and fugitive ink (i.e., material that evacuates post-fabrication to leave vacant layers), and (V3) alternating wide layers of solid HUVEC-laden and solid hPC-laden hydrogel, without hierarchical branching.

FIG. 22 illustrates the mouse model surgical strategy. The MAS is wrapped around a femur bone segment to provide new blood supply and promote healing.

FIGS. 23-25 are photos showing example extrusion nozzles, including coaxial nozzles for coextruding bioink compositions and optionally fugitive ink compositions with a crosslinking agent (FIG. 23), fan-shaped nozzles for extruding sheets (FIG. 24), and curving fan-shaped nozzles (FIG. 25).

### DETAILED DESCRIPTION

The materials, compounds, compositions, systems, and methods described herein may be understood more readily by reference to the following detailed description of specific aspects of the disclosed subject matter and the Examples and Figures included therein.

Before the present materials, compounds, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

In this specification and in the claims which follow, reference will be made to a number of terms which shall be defined to have the following meanings:

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

“Optional” or “optionally” means that the subsequently described event or circumstance may or may not occur, and that the description includes instances where said event or circumstance occurs and instances where it does not.

Provided herein are compositions, systems, and methods for preparing scaffolds for cell or tissue culture. The scaffolds can be non-filamentous, meaning that the scaffold has a form or structure other than a cylindrical, extended wire or fiber. For example, the scaffolds can be in the form of a sheet, a curved sheet, a hollow tube, or a multilayer sheet. These methods can comprise providing at least a first printing composition and a second printing composition; chaotic printing the first printing composition and the second printing composition to generate a microstructured precursor comprising a plurality of lamellar structures formed from the first printing composition and the second printing composition; extruding the microstructured precursor through a nozzle to produce a non-filamentous microstructured precursor; and curing the non-filamentous microstructured precursor to provide the non-filamentous scaffold for cell or tissue culture.

In some embodiments, chaotic printing can comprise a continuous process. In other embodiments, chaotic printing can comprise a batch process.

In some embodiments, chaotic printing of the first printing composition and the second printing composition can comprise inducing laminar flow of the first printing composition and the second printing composition through a mixer that chaotically mixes the first printing composition and the second printing composition to form lamellar interfaces between the first printing composition and the second printing composition.

In some embodiments, chaotic printing of the first printing composition and the second printing composition can comprise coextruding the first printing composition and the second printing composition through a mixer that chaotically mixes the first printing composition and the second printing composition to form lamellar interfaces between the first printing composition and the second printing composition.

In some embodiments, the mixer can comprise a static mixer, such as a Kenics static mixer.

In these embodiments, the mixer can comprise a static mixer, such as a Kenics static mixer (KSM). In some embodiments, the KSM can comprise at least two KSM elements (e.g., at least 3 KSM elements, at least 4 KSM elements, at least 5 KSM elements, at least 6 KSM elements, at least 7 KSM elements, at least 8 KSM elements, or at least 9 KSM elements). In some embodiments, the KSM can comprise 10 KSM elements or less (e.g., 9 KSM elements or less, 8 KSM elements or less, 7 KSM elements or less, 6 KSM elements or less, 5 KSM elements or less, 4 KSM elements or less, or 3 KSM elements or less).

The KSM can comprise a number of KSM elements ranging from any of the minimum values described above to any of the maximum values described above. For example, in some embodiments, the KSM can comprise from 2 to 10 KSM elements (e.g., from 2 to 7 KSM elements, or from 2 to 6 KSM elements).

In some embodiments, chaotic printing of the first printing composition and the second printing composition can comprise coextruding the first printing composition and the second printing composition with a crosslinking agent. In some examples, the first printing composition comprises an alginate and the crosslinking agent comprises a divalent cation. In certain examples, the crosslinking agent comprises a calcium salt such as calcium chloride.

In some embodiments, the nozzle exhibits a substantially non-circular cross-section. In some embodiments, the non-filamentous microstructured precursor and the non-filamentous scaffold exhibit a substantially non-circular cross-section perpendicular to an axis along which extrusion occurs.

In some examples, the nozzle can comprise a fan-shaped nozzle. In these embodiments, the non-filamentous microstructured precursor and the non-filamentous scaffold for cell or tissue culture can comprise a sheet or multilayer sheet. In some embodiments, the sheet can have a width and a height, and the width of the sheet can be at least five times (or at least ten times) the height of the sheet.

In other examples, the nozzle can comprise a curved fan-shaped nozzle or annular nozzle. In these embodiments, the non-filamentous microstructured precursor and the non-filamentous scaffold for cell or tissue culture can comprise a curved sheet or hollow tube.

In some embodiments, the method can further comprise using a multiplexer to select various chaotically printed microstructured precursors that are co-extruded to produce the non-filamentous microstructured precursor.

Once formed, the non-filamentous microstructured precursor (e.g., a bioink composition present in the microstructured precursor) can be cured. Suitable curing methods can be selected based on the identity of the one or more polymers present in the bioink composition. For example, in some examples, the bioink composition can comprise a polymer (e.g., alginate) which crosslinks upon exposure to a metal cation, such as Ca²⁺. In these examples, curing can comprise contacting the non-filamentous microstructured precursor with an aqueous solution comprising metal cations (e.g., Ca²⁺ ions). In other examples, the bioink composition can comprise one or more polymers that comprise an ethylenically unsaturated moiety. In these examples, curing can comprise exposing the non-filamentous microstructured precursor to UV light. In some embodiments, curing can comprise incubating the non-filamentous microstructured precursor (e.g., for a period of time effective for physical crosslinking of polymer.

In some embodiments, the first printing composition comprises a bioink composition.

In some embodiments, the second printing composition also comprises a bioink composition. In some of these examples, the first printing composition and the second printing composition are of different composition.

In other embodiments, the second printing composition comprises a fugitive ink composition. In these embodiments, the methods can further comprise removing the fugitive ink composition from the non-filamentous scaffold following curing.

In some embodiments, the method can further comprise bioprinting, electrospinning, and/or melt electrowriting a third printing composition onto or into the non-filamentous scaffold. In some examples, the third printing composition can comprise a bioink composition. In some of these examples, the third printing composition can be of a different composition than the first printing composition and the second printing composition.

In some embodiments, each of the bioink compositions individually comprises a polymer. In some examples, the polymer can comprise a hydrogel-forming agent. In some examples, the polymer can comprise a polysaccharide, such as alginate, hyaluronic acid, agarose, or any combination thereof. In some examples, the polymer can comprise a protein or peptide, such as gelatin, collagen, or any combination thereof. In some examples, the polymer can comprise a synthetic polymer, such as a polyester (e.g., poly(propylene fumarate) (PPF), polycaprolactone, poly(lactic-co-glycolic acid), polylactic acid, polyglycolic acid, or any combination thereof). In some examples, the polymer is crosslinkable. In some examples, the polymer can be present in an amount of from 0.5% to 20% by weight, based on the total weight of the bioink composition. In some embodiments, the bioink composition can further comprise cells (e.g., pluripotent stem cells, multipotent stem cells, progenitor cells, terminally differentiated cells, endothelial cells, endothelial progenitor cells, immortalized cell lines, primary cells, or any combination thereof).

In certain embodiments, the bioink composition can exhibit a viscosity of less than 1000 cP at 23° C. prior to curing. For example, in some embodiments, the bioink composition can exhibit a viscosity of less than 500 cP, less than 250 cP, or less than 100 cP at 23° C. prior to curing. Upon curing, the bioink composition can increase in viscosity to form a matrix that exhibits a viscosity of at least 25,000 cP at 37° C. (e.g., a viscosity of from 25,000 cP to 100,000 cP at 37° C.).

In some embodiments, the method can further comprise dispersing a population of cells in the bioink composition (e.g., the first printing composition and/or the second printing composition, when the second printing composition comprises a bioink) prior to the chaotic printing. In some embodiments, the method further comprises seeding the non-filamentous scaffold with a population of cells. In these embodiments, the cells can comprise, for example, pluripotent stem cells, multipotent stem cells, progenitor cells, terminally differentiated cells, endothelial cells, endothelial progenitor cells, immortalized cell lines, primary cells, or any combination thereof.

In some embodiments, the fugitive ink composition, when present, comprises a polymer. In some examples, the polymer can comprise a poly(alkylene oxide) block copolymer, such as a polyoxyethylene-polyoxypropylene (PEO-PPO) block copolymers (e.g., a poloxamer). In some examples, the polymer can comprise hydroxyethyl cellulose (HEC). In some examples, the polymer can be present in an amount of from 0.5% to 20% by weight, based on the total weight of the fugitive ink composition.

In certain embodiments, the fugitive ink composition can exhibit a viscosity of less than 1000 cP at 23° C. prior to curing. For example, in some embodiments, the fugitive ink composition can exhibit a viscosity of less than 500 cP, less than 250 cP, or less than 100 cP at 23° C. prior to curing. Upon curing, the fugitive ink composition can retain a viscosity of less than 5,000 cP at 23° C. (e.g., a viscosity of less than 1000 cP, less than 500 cP, less than 250 cP, or less than 100 cP at 23° C.).

Following crosslinking, the fugitive ink can be removed from the cured scaffold precursor. The fugitive ink can be removed by any suitable method. In some embodiments, the fugitive ink can be heated and/or incubated under reduced pressure to drive off the fugitive ink. In other embodiments, the cured scaffold can be immersed in an aqueous solution and/or dialyzed against an aqueous solution to remove the fugitive ink by diffusion. In other embodiments, the cured scaffold can be perfused with an aqueous solution to remove the fugitive ink from within the cured scaffold. Combinations of these methods can also be employed.

In some embodiments, the resulting scaffolds can exhibit an average striation thickness of at least 10 nm (e.g., at least 25 nm, at least 50 nm, at least 75 nm, at least 100 nm, at least 150 nm, at least 200 nm, at least 250 nm, at least 300 nm, at least 400 nm, at least 500 nm, at least 600 nm, at least 700 nm, at least 750 nm, at least 800 nm, at least 900 nm, at least 1 μm, at least 5 μm, at least 10 μm, at least 20 μm, at least 25 μm, at least 30 μm, at least 40 μm, at least 50 μm, at least 100 μm, at least 200 μm, at least 250 μm, at least 300 μm, or at least 400 μm). In some embodiments, the scaffolds can exhibit an average striation thickness of 500 μm or less (e.g., 400 μm or less, 300 μm or less, 250 μm or less, 200 μm or less, 100 μm or less, 50 μm or less, 40 μm or less, 30 μm or less, 25 μm or less, 20 μm or less, 10 μm or less, 5 μm or less, 1 μm or less, 900 nm or less, 800 nm or less, 750 nm or less, 700 nm or less, 600 nm or less, 500 nm or less, 400 nm or less, 300 nm or less, 250 nm or less, 200 nm or less, 150 nm or less, 100 nm or less, 75 nm or less, 50 nm or less, or 25 nm or less).

The scaffolds can exhibit an average striation thickness ranging from any of the minimum values described above to any of the maximum values described above. For example, in some embodiments, the scaffolds can exhibit an average striation thickness of from 10 nm to 500 μm (e.g., from 10 nm to 200 μm, or from 10 nm to 50 μm).

In other embodiments, the scaffolds can include larger striation thicknesses (e.g., striation thicknesses on the millimeter and/or centimeter length scales, such as from 1 mm to 50 cm, or from 1 mm to 10 cm).

In some embodiments, the resulting scaffolds can exhibit a surface-area-to-volume (SAV) of at least 400 m⁻¹ (e.g., at least 500 m⁻¹, at least 600 m⁻¹, at least 700 m⁻¹, at least 750 m⁻¹, at least 800 m⁻¹, at least 900 m⁻¹, at least 1000 m⁻¹, at least 1250 m⁻¹, at least 1500 m⁻¹, at least 1750 m⁻¹, at least 2000 m⁻¹, at least 2250 m⁻¹, at least 2500 m⁻¹, at least 2750 m 1, at least 3000 m²¹, at least 3250 m⁻¹, at least 3500 m⁻¹, at least 3750 m 1, at least 4000 m⁻¹, at least 4250 m⁻¹, at least 4500 m⁻¹, or at least 1750 m⁻¹). In some embodiments, the scaffolds can exhibit a surface-area-to-volume (SAV) of 5000 m⁻¹ or less (e.g., 4750 m⁻¹ or less, 4500 m⁻¹ or less, 4250 m⁻¹ or less, 4000 m⁻¹ or less, 3750 m⁻¹ or less, 3500 m⁻¹ or less, 3250 m⁻¹ or less, 3000 m⁻¹ or less, 2750 m⁻¹ or less, 2500 m⁻¹ or less, 2250 m⁻¹ or less, 2000 m⁻¹ or less, 1750 m⁻¹ or less, 1500 m⁻¹ or less, 1250 m⁻¹ or less, 1000 m⁻¹ or less, 900 m⁻¹ or less, 800 m⁻¹ or less, 750 m⁻¹ or less, 700 m⁻¹ or less, 600 m⁻¹ or less, or 500 m⁻¹ or less).

The scaffolds can exhibit a surface-area-to-volume (SAV) ranging from any of the minimum values described above to any of the maximum values described above. For example, in some embodiments, the scaffolds can exhibit a surface-area-to-volume (SAV) of from 400 m⁻¹ to 5000 m⁻¹.

In some embodiments, the resulting scaffold can exhibit a surface density of at least 0.05 m² cm⁻³ (at least 0.055 m² cm⁻³, at least 0.06 m² cm⁻³, at least 0.065 m² cm⁻³, at least 0.07 m² cm⁻³, at least 0.075 m² cm⁻³, or more),

### Bioink Compositions

The bioink composition can comprise an aqueous solution comprising one or more polymers (e.g., one or more biopolymers). Following processing, the bioink will form the laminae of the microstructured scaffolds described herein. Accordingly, the one or more polymers can be selected and included in an amount effective such that the polymers form biocompatible laminae suitable to support cell culture upon curing. In some embodiments, the one or more polymers can be biodegradable.

In certain embodiments, the one or more polymers can comprise a hydrogel-forming agent. The term “hydrogel” refers to a broad class of polymeric materials, that may be natural or synthetic, which have an affinity for an aqueous medium, and may absorb large amounts of the aqueous medium, but which do not normally dissolve in the aqueous medium. Generally, a hydrogel may be formed by using at least one, or one or more types of hydrogel-forming agent, and setting or solidifying the one or more types of hydrogel-forming agent in an aqueous medium to form a three-dimensional network, wherein formation of the three-dimensional network may cause the one or more types of hydrogel-forming agent to gel so as to form the hydrogel. The term “hydrogel-forming agent”, also termed herein as “hydrogel precursor”, refers to any chemical compound that may be used to make a hydrogel. The hydrogel forming agent may comprise a physically cross-linkable polymer, a chemically cross-linkable polymer, or mixtures thereof.

Physical crosslinking may take place via, for example, complexation, hydrogen bonding, desolvation, van der Waals interactions, or ionic bonding. In various embodiments, a hydrogel may be formed by self-assembly of one or more types of hydrogel-forming agents in an aqueous medium. The term “self-assembly” refers to a process of spontaneous organization of components of a higher order structure by reliance on the attraction of the components for each other, and without chemical bond formation between the components. For example, polymer chains may interact with each other via any one of hydrophobic forces, hydrogen bonding, Van der Waals interaction, electrostatic forces, or polymer chain entanglement, induced on the polymer chains, such that the polymer chains aggregate or coagulate in an aqueous medium to form a three-dimensional network, thereby entrapping molecules of water to form a hydrogel. Examples of physically cross-linkable polymer that may be used include, but are not limited to, gelatin, alginate, pectin, furcellaran, carageenan, chitosan, derivatives thereof, copolymers thereof, and mixtures thereof.

Chemical crosslinking refers to an interconnection between polymer chains via chemical bonding, such as, but not limited to, covalent bonding, ionic bonding, or affinity interactions (e.g. ligand/receptor interactions, antibody/antigen interactions, etc.). Examples of chemically cross-linkable polymer that may be used include, but are not limited to, starch, gellan gum, dextran, hyaluronic acid, poly(ethylene oxides), polyphosphazenes, derivatives thereof, copolymers thereof, and mixtures thereof. Other suitable polymers include polymers (gelatin, cellulose, etc.) functionalized with ethylenically unsaturated moieties (e.g., (meth)acrylate groups). Such polymers may be cross-linked in site via polymerization of these groups. An example of such a material is gelatin methacrylate (GelMA), which is denatured collagen that is modified with photopolymerizable methacrylate (MA) groups.

Optionally, chemical cross-linking may take place in the presence of a chemical cross-linking agent. The term “chemical cross-linking agent” refers to an agent which induces chemical cross-linking. The chemical cross-linking agent may be any agent that is capable of inducing a chemical bond between adjacent polymeric chains. For example, the chemical cross-linking agent may be a chemical compound. Examples of chemical compounds that may act as cross-linking agent include, but are not limited to, 1-ethyl-3-[3-dimethylaminopropyl]carbodiimide hydrochloride (EDC), vinylamine, 2-aminoethyl methacrylate, 3-aminopropyl methacrylamide, ethylene diamine, ethylene glycol dimethacrylate, methylmethacrylate, N,N′ methylene-bisacrylamide, N,N′-methylene-bis-methacrylamide, diallyltartardiamide, allyl(meth)acrylate, lower alkylene glycol di(meth)acrylate, poly lower alkylene glycol di(meth)acrylate, lower alkylene di(meth)acrylate, divinyl ether, divinyl sulfone, di- or trivinylbenzene, trimethylolpropane tri(meth)acrylate, pentaerythritol tetra(meth)acrylate, bisphenol A di(meth)acrylate, methylenebis(meth)acrylamide, triallyl phthalate, diallyl phthalate, transglutaminase, derivatives thereof or mixtures thereof. However, in some embodiments, the hydrogel-forming agents are themselves capable of chemical or physical cross-linking without using a cross-linking agent.

Besides the above-mentioned, the hydrogel-forming agents may be cross-linked using a cross-linking agent in the form of an electromagnetic wave. The cross-linking may be carried out using an electromagnetic wave, such as gamma or ultraviolet radiation, which may cause the polymeric chains to cross-link and form a three-dimensional matrix, thereby entrapping water molecules to form a hydrogel.

In some embodiments, the one or more polymers can comprise a natural polymer. A “natural polymer” refers a polymeric material that may be found in nature. In various embodiments, examples of such natural polymers include polysaccharides, glycosaminoglycans, proteins, and mixtures thereof.

Polysaccharides are carbohydrates which may be hydrolyzed to two or more monosaccharide molecules. They may contain a backbone of repeating carbohydrate i.e. sugar unit. Examples of polysaccharides include, but are not limited to, alginate, agarose, chitosan, dextran, starch, gellan gum, and mixtures thereof. Glycosaminoglycans are polysaccharides containing amino sugars as a component. Examples of glycosaminoglycans include, but are not limited to, hyaluronic acid, chondroitin sulfate, dermatin sulfate, keratin sulfate, dextran sulfate, heparin sulfate, heparin, glucuronic acid, iduronic acid, galactose, galactosamine, and glucosamine.

Peptides, which form building blocks of polypeptides and in turn proteins, generally refer to short chains of amino acids linked by peptide bonds. Typically, peptides comprise amino acid chains of about 2-100, more typically about 4-50, and most commonly about 6-20 amino acids. Polypeptides generally refer to individual straight or branched chain sequences of amino acids that are typically longer than peptides. They usually comprise at least about 20 to 1000 amino acids in length, more typically at least about 100 to 600 amino acids, and frequently at least about 200 to about 500 amino acids. Included are homo-polymers of one specific amino acid, such as for example, poly-lysine. Proteins include single polypeptides as well as complexes of multiple polypeptide chains, which may be the same or different.

Proteins have diverse biological functions and can be classified into five major categories, i.e. structural proteins such as collagen, catalytic proteins such as enzymes, transport proteins such as hemoglobin, regulatory proteins such as hormones, and protective proteins such as antibodies and thrombin. Other examples of proteins include, but are not limited to, fibronectin, gelatin, fibrin, pectins, albumin, ovalbumin, and polyamino acids.

In other embodiments, the one or more polymers can comprise a synthetic polymer. Examples of suitable synthetic polymers include, for example, a polyester such as poly(propylene fumarate) (PPF), polylactic acid (PLA), polyglycolic acid (PGA), poly lactic-co-glycolide (PLGA), polycaprolactone (PCL), polydioxanone (PDS), a polyhydroxyalkanoate (PHA), a polyurethane (PU), copolymers thereof, and blends thereof. Examples of polyhydroxyalkanoates include poly-3-hydroxybutyrate (P3HB), poly-4-hydroxybutyrate (P4HB), polyhydroxyvalerate (PHV), polyhydroxybexanoate (PHH), polyhydroxyoctanoate (PHO), copolymers thereof, and blends thereof. Other suitable biodegradable synthetic polymers include, for example, polyurethanes. In certain embodiments, the biodegradable synthetic polymer can comprise PGA.

In some embodiments, the one or more polymers can comprise alginate, agarose, or a combination thereof. In some embodiments, the one or more polymers can comprise alginate. The term “alginate” refers to any of the conventional salts of algin, which is a polysaccharide of marine algae, and which may be polymerized to form a matrix for use in drug delivery and in tissue engineering due to its biocompatibility, low toxicity, relatively low cost, and simple gelation with divalent cations such as calcium ions (Ca²⁺) and magnesium ions (Mg²⁺). Examples of alginate include sodium alginate which is water soluble, and calcium alginate which is insoluble in water. In some embodiments, the one or more polymers can comprise agarose. Agarose refers to a neutral gelling fraction of a polysaccharide complex extracted from the agarocytes of algae such as a Rhodophycede.

In some embodiments, the one or more polymers can comprise gelatin. The term “gelatin” as used herein refers to protein substances derived from collagen. In the context of this description, “gelatin” also refers to equivalent substances such as synthetic analogues of gelatin (e.g., gelatin methacrylate (GelMA)). Generally, gelatin may be classified as alkaline gelatin, acidic gelatin, or enzymatic gelatin. Alkaline gelatin may be obtained from the treatment of collagen with a base such as sodium hydroxide or calcium hydroxide. Acidic gelatin may be obtained from the treatment of collagen with an acid such as hydrochloric acid. Enzymatic gelatin may be obtained from the treatment of collagen with an enzyme such as hydrolase.

In certain embodiments, the bioink composition can comprise collagen, hyaluronate, fibrin, alginate, agarose, chitosan, gelatin, matrigel, glycosaminoglycans, or a combination thereof.

In some embodiments, the one or more polymers can be present in an amount of at least 0.5% by weight (e.g., at least 1.0% by weight, at least 1.5% by weight, at least 2.0% by weight, at least 2.5% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, at least 16% by weight, at least 17% by weight, at least 18% by weight, or at least 19% by weight), based on the total weight of the bioink composition. In some embodiments, the one or more polymers can be present in an amount of 20% by weight or less (e.g., 19% by weight or less, 18% by weight or less, 17% by weight or less, 16% by weight or less, 15% by weight or less, 14% by weight or less, 13% by weight or less, 12% by weight or less, 11% by weight or less, 10% by weight or less, 9% by weight or less, 8% by weight or less, 7% by weight or less, 6% by weight or less, 5% by weight or less, 4% by weight or less, 3% by weight or less, 2.5% by weight or less, 2% by weight or less, 1.5% by weight or less, or 1% by weight or less), based on the total weight of the bioink composition.

The amount of the one or more polymers present in the bioink composition can be present in an amount ranging from any of the minimum values described above to any of the maximum values described above. For example, in some embodiments, the one or more polymers can be present in the bioink composition in an amount of from 0.5% to 20% by weight, based on the total weight of the bioink composition.

In certain examples, the bioink composition can comprise from 2% by weight to 10% by weight gelatin methacrylate (GelMA) and from 1% by weight to 4% by weight alginate.

In some embodiments, the bioink composition can further comprise a population of cells, one or more bioactive agents, or any combination thereof (as described in more detail below).

The bioink composition can include one or more polymers dissolved in an aqueous medium to form a solution. The terms “aqueous medium” and “aqueous solution” as used herein are used interchangeably, and refers to water or a solution based primarily on water such as phosphate buffered saline (PBS), or water containing a salt dissolved therein. The aqueous medium may also comprise or consist of a cell culture medium. The term “cell culture medium” refers to any liquid medium which enables cells proliferation. Growth media are known in the art and can be selected depending of the type of cell to be grown. For example, a growth medium for use in growing mammalian cells is Dulbecco's Modified Eagle Medium (DMEM) which can be supplemented with heat inactivated fetal bovine serum.

The bioink composition can be prepared by dissolving one or more polymers in an aqueous medium to form a solution. Agitation, for example, by stirring or sonication may be carried out to enhance the rate at which the one or more polymers dissolve in the aqueous medium. In some cases, heat energy may optionally be applied to the aqueous medium to increase the dissolution rate of the one or more polymers in the aqueous medium.

In some embodiments, the bioink can further include a population of nanoparticles, a population of microparticles, or a combination thereof. In some embodiments, the microparticles and nanoparticles can comprise polymer particles. The polymer particles can be formed from polylactides (e.g., poly(lactic acid) (PLA), poly(lactic-co-glycolic acid) (PLGA), and poly(lactic acid)-polyethyleneglycol (PLA-PEG) block copolymers), polyesters (e.g., polycaprolactone and polyhydroxyalkanoates such as poly-3-hydroxybutyrate (PHB) and poly-4-hydroxybutyrate (P4HB)), polyglycolides, polyanhydrides, poly(ester anhydrides), polyalkylene oxides (e.g., polyethylene glycols, polypropylene glycols, polybutylene glycols, and copolymers thereof), polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, poly(amino acids), cellulosic polymers (e.g., cellulose and derivatives thereof, such as hydroxypropyl methyl cellulose, ethyl cellulose, methyl cellulose, sodium carboxymethyl cellulose (NaCMC), and polyhydroxycellulose), dextrans, gelatin, chitin, chitosan, alginates, hyaluronic acid, as well as copolymers (random copolymers as well as block copolymers), terpolymers and mixtures thereof.

In some embodiments, one or more bioactive agents (as discussed below) can be conjugated to the surface of the particles. In some embodiments, one or more bioactive agents can be dispersed or encapsulated within the particles. In these embodiments, the particles can provide for the controlled or sustained release of one or more bioactive agents within the laminae over time.

The bioink composition can optionally include one or more additional components, such as a photoinitiator, solvent, surfactant, light attenuator, crosslinker, nutrient, or any combination thereof. In certain examples, the bioink composition can further comprise a photoinitator to facilitate curing.

### Fugitive Ink Compositions

The fugitive ink composition can comprise an aqueous solution comprising one or more polymers which can be readily removed at some point following curing. In some embodiments, the one or more polymers are not crosslinkable or otherwise curable under conditions used to cure the bioink composition. In other embodiments, the one or more polymers can be crosslinkable or otherwise curable, but form a much less robust polymer network upon curing than the cured bioink composition. For example, the one or more polymers present in the fugitive ink composition can initially crosslink or otherwise cure to form fugitive layers following curing. The fugitive layers can then be readily removed while leaving the cured bioink layers (laminae) intact. For example, in some embodiments, the fugitive layer can degrade over time, such that the fugitive layers can removed some period of time following curing. In other examples, the fugitive layers can decay or dissolve in response to a stimulus (e.g., irradiation with light, heat, contact with an enzyme, or exposure to an acid or base), allowing for removal of the fugitive layers at a desired point following curing.

Examples of suitable polymers include, but are not limited to, polylactides (e.g., poly(lactic acid) (PLA), poly(lactic-co-glycolic acid) (PLGA), and poly(lactic acid)-polyethyleneglycol (PLA-PEG) block copolymers), polyesters (e.g., polycaprolactone and polyhydroxyalkanoates such as poly-3-hydroxybutyrate (PHB) and poly-4-hydroxybutyrate (P4HB)), polyglycolides, polyanhydrides, poly(ester anhydrides), polyalkylene oxides (e.g., polyethylene glycols, polypropylene glycols, polybutylene glycols, and copolymers thereof), polyamines, polyurethanes, polyesteramides, polyorthoesters, polydioxanones, polyacetals, polyketals, polycarbonates, polyphosphoesters, polyoxaesters, polyorthocarbonates, polyphosphazenes, succinates, poly(malic acid), poly(amino acids), polyvinylpyrrolidone, polyethylene glycol, poly(amino acids), cellulosic polymers (e.g., cellulose and derivatives thereof, such as hydroxypropyl methyl cellulose, ethyl cellulose, methyl cellulose, sodium carboxymethyl cellulose (NaCMC), and polyhydroxycellulose), dextrans, gelatin, chitin, chitosan, alginates, hyaluronic acid, as well as copolymers (random copolymers as well as block copolymers), terpolymers and mixtures thereof.

In some examples, the one or more polymers can comprise a polylactide, that is, a lactic acid-based polymer that can be based solely on lactic acid or can be a copolymer based on lactic acid, glycolic acid, and/or caprolactone, which may include small amounts of other comonomers. As used herein, the term “lactic acid” includes the isomers L-lactic acid, D-lactic acid, DL-lactic acid and lactide, while the term “glycolic acid” includes glycolide. Examples include polymers selected from the group consisting of polylactide polymers, commonly referred to as PLA, poly(lactide-co-glycolide) copolymers, commonly referred to as PLGA, and poly(caprolactone-co-lactic acid) (PCL-co-LA). In some examples, the polymer may have a monomer ratio of lactic acid/glycolic acid of from about 100:0 to about 15:85, preferably from about 75:25 to about 30:70, more preferably from about 60:40 to about 40:60, and an especially useful copolymer has a monomer ratio of lactic acid/glycolic acid of about 50:50.

The poly(caprolactone-co-lactic acid) (PCL-co-LA) polymer can have a comonomer ratio of caprolactone/lactic acid of from about 10:90 to about 90:10, from about 35:65 to about 65:35; or from about 25:75 to about 75:25. In certain embodiments, the lactic acid based polymer comprises a blend of about 0% to about 90% caprolactone, about 0% to about 100% lactic acid, and about 0% to about 60% glycolic acid.

The lactic acid-based polymer can have a number average molecular weight of from about 1,000 to about 120,000 (e.g., from about 5,000 to about 50,000, or from about 8,000 to about 30,000), as determined by gel permeation chromatography (GPC). Suitable lactic acid-based polymers are available commercially. For instance, 50:50 lactic acid:glycolic acid copolymers having molecular weights of 8,000, 10,000, 30,000 and 100,000 are available from Boehringer Ingelheim (Petersburg, Va.), Medisorb Technologies International L.P. (Cincinatti, Ohio) and Birmingham Polymers, Inc. (Birmingham, Ala.) as described below.

Examples of other suitable polymers include, but are not limited to, Poly (D,L-lactide) Resomer® L104, PLA-L104, Poly (D,L-lactide-co-glycolide) 50:50 Resomer® RG502, Poly (D,L-lactide-co-glycolide) 50:50 Resomer® RG502H, Poly (D,L-lactide-co-glycolide) 50:50 Resomer® RG503, Poly (D,L-lactide-co-glycolide) 50:50 Resomer® RG506, Poly L-Lactide MW 2,000 (Resomer® L 206, Resomer® L 207, Resomer® L 209, Resomer® L 214); Poly D,L Lactide (Resomer® R 104, Resomer® R 202, Resomer® R 203, Resomer® R 206, Resomer® R 207, Resomer® R 208); Poly L-Lactide-co-D,L-lactide 90:10 (Resomer® LR 209); Poly glycolide (Resomer® G 205); Poly D,L-lactide-co-glycolide 50:50 (Resomer® RG 504 H, Resomer® RG 504, Resomer® RG 505); Poly D-L-lactide-co-glycolide 75:25 (Resomer® RG 752, Resomer® RG755, Resomer® RG 756); Poly D,L-lactide-co-glycolide 85:15 (Resomer® RG 858); Poly L-lactide-co-trimethylene carbonate 70:30 (Resomer® LT 706); Poly dioxanone (Resomer® X 210) (Boehringer Ingelheim Chemicals, Inc., Petersburg, Va.).

Additional examples include, but are not limited to, DL-lactide/glycolide 100:0 (MEDISORB® Polymer 100 DL High, MEDISORB® Polymer 100 DL Low); DL-lactide/glycolide 85/15 (MEDISORB® Polymer 8515 DL High, MEDISORB® Polymer 8515 DL Low); DL-lactide/glycolide 75/25 (MEDISORB® Polymer 7525 DL High, MEDISORB® Polymer 7525 DL Low); DL-lactide/glycolide 65/35 (MEDISORB® Polymer 6535 DL High, MEDISORB® Polymer 6535 DL Low); DL-lactide/glycolide 54/46 (MEDISORB® Polymer 5050 DL High, MEDISORB® Polymer 5050 DL Low); and DL-lactide/glycolide 54/46 (MEDISORB® Polymer 5050 DL 2A (3), MEDISORB® Polymer 5050 DL 3A (3), MEDISORB® Polymer 5050 DL 4A (3)) (Medisorb Technologies International L.P., Cincinati, Ohio); and Poly D,L-lactide-co-glycolide 50:50; Poly D,L-lactide-co-glycolide 65:35; Poly D,L-lactide-co-glycolide 75:25; Poly D,L-lactide-co-glycolide 85:15; Poly DL-lactide; Poly L-lactide; Poly glycolide; Poly ¿-caprolactone; Poly DL-lactide-co-caprolactone 25:75; and Poly DL-lactide-co-caprolactone 75:25 (Birmingham Polymers, Inc., Birmingham, Ala.).

In some examples, the one or more polymers can comprise a biodegradable, biocompatible poly(alkylene oxide) block copolymer, such as a block copolymer of polyethylene oxide and polypropylene oxide (also referred to as poloxamers). Examples of polyoxyethylene-polyoxypropylene (PEO-PPO) block copolymers include PLURONIC® F127 and F108, which are PEO-PPO block copolymers with molecular weights of 12,600 and 14,600, respectively. Each of these compounds is available from BASF of Mount Olive, N.J. PLURONIC® acid F127 in PBS.

In some examples, the one or more polymers can comprise block polymers such as polyoxyethylene-polyoxypropylene (PEO-PPO) block polymers of the general structure A-B, (A-B) n, A-B-A (e.g., a poloxamer or PLURONIC®), or (A-B-A), with A being the PEO part and B being the PPO part and n being greater than 1. In other embodiments, the one or more polymers can comprise branched polymers of polyoxyethylene-polyoxypropylene (PEO-PPO) like tetra-functional poloxamines (e.g., a poloxamine or TETRONIC®). For example, the one or more polymers can comprise poloxamer 407, poloxamer 188, poloxamer 234, poloxamer 237, poloxamer 338, poloxamine 1107, poloxamine 1307, or a combination thereof.

Advantageously, poloxamers have surfactant abilities and extremely low toxicity and immunogenic responses. Thus, traces of poloxamers following removal of the fugitive ink can exhibit minimal impact on cells present in the bioink composition and/or cells subsequently seeded into the scaffold.

The average molecular weights of the poloxamers can range from about 1,000 to greater than 16,000 Daltons. Because the poloxamers are products of a sequential series of reactions, the molecular weights of the individual poloxamer molecules form a statistical distribution about the average molecular weight. In addition, commercially available poloxamers can contain substantial amounts of poly(oxyethylene) homopolymer and poly(oxyethylene)/poly(oxypropylene diblock polymers. The relative amounts of these byproducts increase as the molecular weights of the component blocks of the poloxamer increase. Depending upon the manufacturer, these byproducts may constitute from about 15 to about 50% of the total mass of the polymer.

In certain embodiments, the fugitive ink composition can comprise hydroxyethyl cellulose (HEC).

In some embodiments, the one or more polymers can be present in an amount of at least 0.5% by weight (e.g., at least 1.0% by weight, at least 1.5% by weight, at least 2.0% by weight, at least 2.5% by weight, at least 3% by weight, at least 4% by weight, at least 5% by weight, at least 6% by weight, at least 7% by weight, at least 8% by weight, at least 9% by weight, at least 10% by weight, at least 11% by weight, at least 12% by weight, at least 13% by weight, at least 14% by weight, at least 15% by weight, at least 16% by weight, at least 17% by weight, at least 18% by weight, or at least 19% by weight), based on the total weight of the fugitive ink composition. In some embodiments, the one or more polymers can be present in an amount of 20% by weight or less (e.g., 19% by weight or less, 18% by weight or less, 17% by weight or less, 16% by weight or less, 15% by weight or less, 14% by weight or less, 13% by weight or less, 12% by weight or less, 11% by weight or less, 10% by weight or less, 9% by weight or less, 8% by weight or less, 7% by weight or less, 6% by weight or less, 5% by weight or less, 4% by weight or less, 3% by weight or less, 2.5% by weight or less, 2% by weight or less, 1.5% by weight or less, or 1% by weight or less), based on the total weight of the fugitive ink composition.

The amount of the one or more polymers present in the fugitive ink composition can be present in an amount ranging from any of the minimum values described above to any of the maximum values described above. For example, in some embodiments, the one or more polymers can be present in the fugitive ink composition in an amount of from 0.5% to 20% by weight, based on the total weight of the fugitive ink composition.

The fugitive ink composition can include one or more polymers dissolved in an aqueous medium to form a solution. The terms “aqueous medium” and “aqueous solution” as used herein are used interchangeably, and refers to water or a solution based primarily on water such as phosphate buffered saline (PBS), or water containing a salt dissolved therein. The aqueous medium may also comprise or consist of a cell culture medium. The term “cell culture medium” refers to any liquid medium which enables cells proliferation. Growth media are known in the art and can be selected depending of the type of cell to be grown. For example, a growth medium for use in growing mammalian cells is Dulbecco's Modified Eagle Medium (DMEM) which can be supplemented with heat inactivated fetal bovine serum.

The fugitive ink composition can be prepared by dissolving one or more polymers in an aqueous medium to form a solution. Agitation, for example, by stirring or sonication may be carried out to enhance the rate at which the one or more polymers dissolve in the aqueous medium. In some cases, heat energy may optionally be applied to the aqueous medium to increase the dissolution rate of the one or more polymers in the aqueous medium.

### Cells

Methods can further comprise incorporating a population of cells within the scaffolds described herein. In some embodiments, methods can further comprise dispersing a population of cells in a bioink composition prior to chaotic printing. As a result, the population of cells can be printed within the scaffold. Such methods can provide for careful control of cell density and position throughout the scaffold. For example, by including a population of cells within the bioink composition, scaffolds can be printed including adjacent layers of different types of cells, layers as thin as one cell thick, and/or layers spaced apart from adjacent layers by controllable distances. Such scaffolds mimic environments observed, for example, within an embryo. As such, the scaffolds can provide in improved environment in which to control, for example, cellular differentiation. In certain embodiments, two or more distinct populations of cells (e.g., two different types of cells can be printed within the scaffold. In other embodiments, the scaffold can be seeded with a population of cells following printing (e.g., by profusion with a fluid containing a population of cells dispersed therein).

The population of cells can include any desired population of viable cells. The viable cells may include any mammalian cell type selected from cells that make up the mammalian body, including germ cells, somatic cells, and stem cells. Depending on the type of cell, cells that make up the mammalian body can be derived from one of the three primary germ cell layers in the very early embryo: endoderm, ectoderm or mesoderm. The term “germ cells” refers to any line of cells that give rise to gametes (eggs and sperm). The term “somatic cells” refers to any biological cells forming the body of a multicellular organism; any cell other than a gamete, germ cell, gametocyte or undifferentiated stem cell.

For example, in mammals, somatic cells make up all the internal organs, skin, bones, blood and connective tissue. As such, a cell may include any somatic cell isolated from mammalian tissue, including organs, skin, bones, blood and connective tissue (i.e., stromal cells). Examples of somatic cells include fibroblasts, chondrocytes, osteoblasts, tendon cells, mast cells, wandering cells, immune cells, pericytes, inflammatory cells, endothelial cells, myocytes (cardiac, skeletal and smooth muscle cells), adipocytes (i.e., lipocytes or fat cells), parenchyma cells (neurons and glial cells, nephron cells, hepatocytes, pancreatic cells, lung parenchyma cells) and non-parenchymal cells (e.g., sinusoidal hepatic endothelial cells, Kupffer cells and hepatic stellate cells). The term “stem cells” refers to cells that have the ability to divide for indefinite periods and to give rise to virtually all of the tissues of the mammalian body, including specialized cells. The stem cells include pluripotent cells, which upon undergoing further specialization become multipotent progenitor cells that can give rise to functional or somatic cells. Examples of stem and progenitor cells include hematopoietic stem cells (adult stem cells; i.e., hemocytoblasts) from the bone marrow that give rise to red blood cells, white blood cells, and platelets; mesenchymal stem cells (adult stem cells) from the bone marrow that give rise to stromal cells, fat cells, and types of bone cells; epithelial stem cells (progenitor cells) that give rise to the various types of skin cells; neural stem cells and neural progenitor cells that give rise to neuronal and glial cells; and muscle satellite cells (progenitor cells) that contribute to differentiated muscle tissue.

In some examples, the cells can comprise pluripotent stem cells, multipotent stem cells, progenitor cells, terminally differentiated cells, endothelial cells, endothelial progenitor cells, immortalized cell lines, primary cells, or any combination thereof.

When added to the bioink composition prior to chaotic printing, the concentration of cells may vary depending on the composition and quantity of the bioink composition. In embodiments, the concentration of cells in the bioink composition may be in the range from about 1×10³ cells ml⁻¹ to about 1×10¹⁰ cells ml⁻¹ of bioink composition, such as about 1×10³ cells ml⁻¹ to about 1×10⁷ cells ml⁻¹, about 1×10⁵ cells ml⁻¹ to about 1×10⁷ cells ml⁻¹, about 1×10⁵ cells ml⁻¹ to about 1×10¹⁰ cells ml⁻¹, about 1×10⁷ cells ml⁻¹ to about 1×10¹⁰ cells ml⁻¹, about 1×10⁵ cells ml⁻¹, about 1×10⁶ cells ml⁻¹, about 1×10⁷ cells ml⁻¹, or about 1×10⁸ cells ml⁻¹.

### Bioactive Agents

In some embodiments, a bioink composition can further include one or more bioactive agents. These bioactive agents can ultimately be incorporated into the laminae of the scaffolds therein. In some embodiments, the bioactive agents can be dissolved or dispersed in a bioink composition. In some embodiments, the bioactive agents can be bioconjugated to one or more polymers present in a bioink composition. In other embodiments, the scaffold can be treated with one or more bioactive agents following synthesis (e.g., by perfusing the scaffold with a solution or suspension comprising one or more bioactive agents). Such an approach may also be used to generate gradients of cues within the scaffold. Cells respond to gradients of fixed and diffusible chemical cues during development, wound healing and inflammatory responses that can direct cell migration, proliferation and differentiation.

As used herein, “bioactive agents” refers to any chemical substances that have an effect in a biological system, whether such system is in vitro, in vivo, or in situ. Examples of classes of bioactive agents include, but are not limited to growth factors, cytokines, antiseptics, antibiotics, anti-inflammatory agents, chemotherapeutic agents, clotting agents, metabolites, chemoattractants, hormones, steroids, morphogens, growth inhibitors, other drugs, or cell attachment molecules.

The term “growth factors” refers to factors affecting the function of cells such as osteogenic cells, fibroblasts, neural cells, endothelial cells, epithelial cells, keratinocytes, chondrocytes, myocytes, cells from joint ligaments, and cells from the nucleus pulposis. Examples of growth factors include platelet derived growth factors (PDGF), the transforming growth factors (TGF-beta), insulin-like growth factors (IGFs), fibroblast growth factors (PGFs). VEGF, EGF, and the bone morphogenetic proteins (BMPs).

The term “cytokines” refers to peptide protein mediators that are produced by immune cells to modulate cellular functions. Examples of cytokines include, but are not limited to, interleukin-1β (IL-1β), interleukin-6 (IL-6), and tumor necrosis factor-α (TNFα).

The term “antiseptics” refers to a chemical agent that inhibits growth of disease-carrying microorganisms. Examples of antiseptics include peroxides, C6-C14 alkyl carboxylic acids and alkyl ester carboxylic acids, antimicrobial natural oils, antimicrobial metals and metal salts such as silver, copper, zinc and their salts.

The term “antibiotic” includes bactericidal, fungicidal, and infection-preventing drugs which are substantially water-soluble such as, for example, gentamicin, vancomycin, penicillin, and cephalosporins. An antibiotic can be added, for example, for selection of the cells or to prevent bacterial growth.

The term “anti-inflammatory agent” refers to any agent possessing the ability to reduce or eliminate cerebral edema (fluid accumulation) or cerebral ischemia, and can include examples such as free radical scavengers and antioxidants, non steroidal anti-inflammatory drugs, steroidal anti-inflammatory agents, stress proteins, or NMDA antagonists.

The term “chemotherapeutic agents” refer to any natural or synthetic molecules that are effective against one or more forms of cancer, and may include molecules that are cytotoxic (anti-cancer agent), simulate the immune system (immune stimulator), or molecules that modulate or inhibit angiogenesis. Examples of chemotherapeutic agents include alkylating agents, antimetabolites, taxanesm, cytotoxics, and cytoprotectant adjuvants.

The term “clotting agent” refers to refers to any molecule or compound that promotes the clotting of blood. Examples of clotting agents include a thrombin agent, which is commonly used as a topical treatment by vascular surgeons to stop surface bleeding after a large surface incision is made in the body, heparin, warfarin, and coumarin derivatives.

The term “metabolite” refers to an intermediate or a product derived from enzymatic conversion of a substrate administered to a subject, the conversion occurring as part of a metabolic process of the subject. Examples of metabolite include glucose, carbohydrates, amino acids and lipids.

The term “chemoattractants” refers to a substance that elicits accumulation of cells, such as chemokines, monocyte chemoattractant protein-1, and galectin-3.

The term “hormone” refers to trace substances produced by various endocrine glands which serve as chemical messengers carried by the blood to various target organs, where they regulate a variety of physiological and metabolic activities in vertebrates. Examples of hormones include steroidal estrogens, progestins, androgens, and the progestational hormone progesterone. Steroids may also be classified as lipids. Naturally occurring steroids are hormones that are important regulators of animal development and metabolism at very low concentrations. Examples of steroids include cholesterol, cortisone, and derivatives of estrogens and progesterones.

The term “cell attachment molecules” as used herein includes, but is not limited to, fibronectin, vitronectin, collagen type I, osteopontin, bone sialoprotein thrombospondin, and fibrinogen. Such molecules are important in the attachment of anchorage-dependent cells.

By way of non-limiting illustration, examples of certain embodiments of the present disclosure are given below.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1: Methods of Using Chaotic Printing to form Thin Membrane Structures

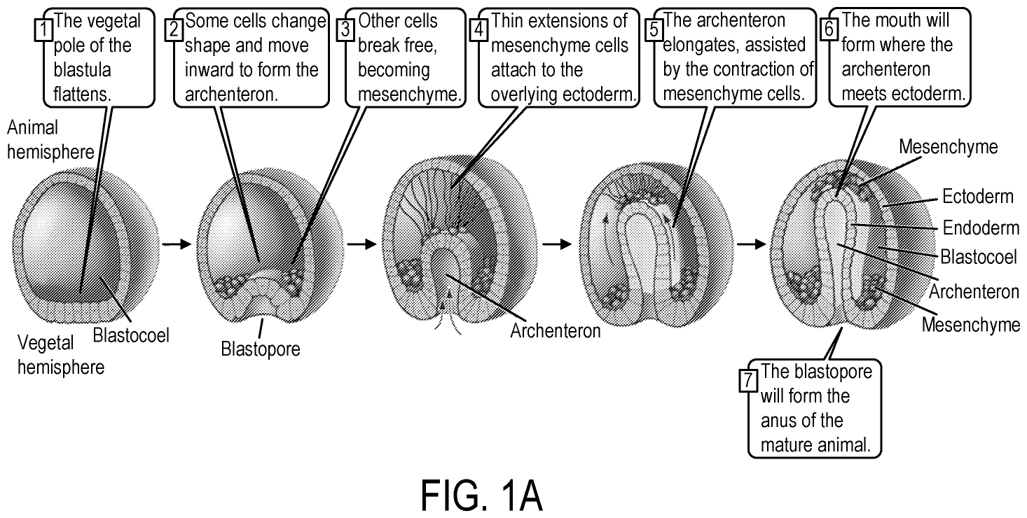

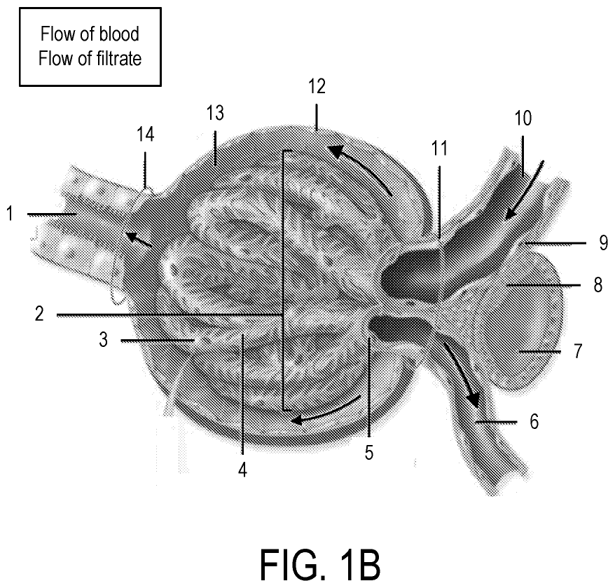

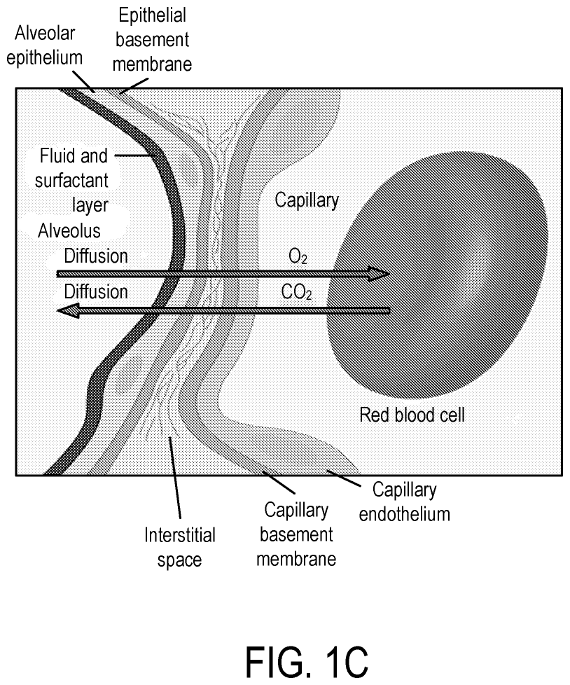

Organ function, embryological or in wound healing, depends initially on the formation of adjacent layers of different types of cells. Vertebrate life begins with the fertilized egg, a single cell that eventually produces a ball of cells, the blastula (FIG. 1A). One could say that gastrulation, the indentation of that ball of cells is the initial bilayer structure where the layers are composed of two different types of cells. Those two layers of apposed cells, the original ectoderm and endoderm layers, “induce” each other to form the third primordial layer, mesoderm. Similarly, two or more layers of different types of cells bring about the formation of the functional portions, the parenchyma, of most organs through cell-cell communication. This process of induction results in the single cell thick layers of the glomerulus of each nephron in the kidney (FIG. 1B), the alveoli in the lung (FIG. 1C), and the discrete physiological or mechanical functions of the parenchyma of most tissues. C.H. Waddington first proposed that embryonic development was due to cells of two different types “inducing” each other through the production of cell signaling molecules (e.g., growth factors, cytokines, and hormones) to bring about new function during growth and development, with some organ function persisting as single cell-thick membranes throughout a vertebrate organism's life.

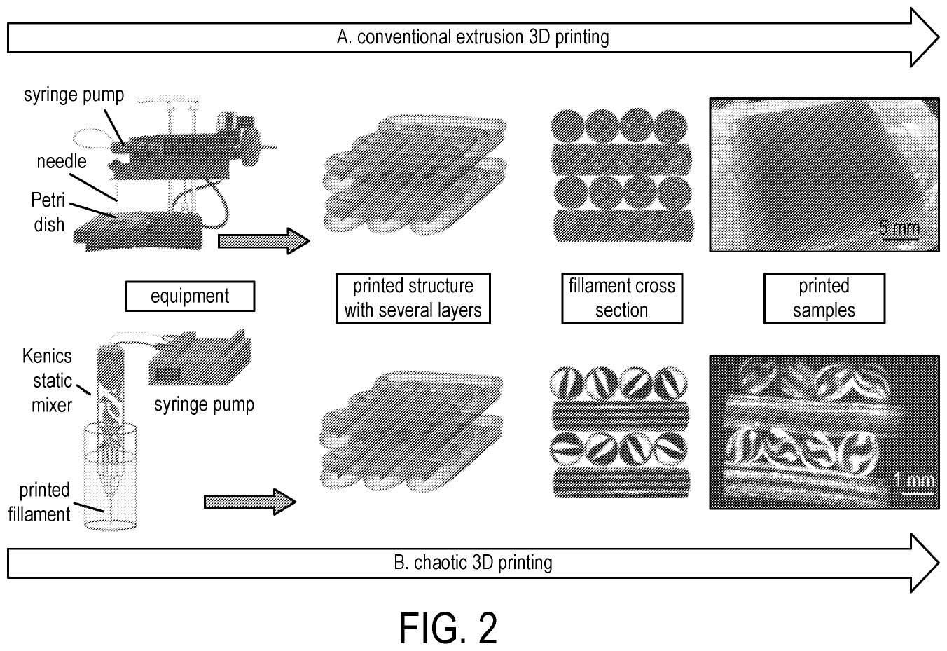

Current nozzle-based biofabrication techniques cannot produce thin membranes (sheets) nor position different types of cells in fully adjacent (contiguous) sheets. Chaotic printing can produce adjacent sheets of cell-laden hydrogels as thin as a single cell and place them near fugitive layers or channels that can facilitate cell media perfusion and/or vascularization. Current bioprinting devices usually consist of a pneumatically or mechanically-driven extruder and a petri dish to collect a cell-laden filament. These filaments are usually thousands to tens of thousands of cells thick (FIG. 2, top right). It is virtually impossible to produce a continuous sheet of these cylindrical fibers that will closely approximate two different types of cells (i.e., two contiguous sheets of cells) by stacking sheets of these filaments (e.g., 1-5 mm diameter filaments). There will be only very small areas, at the apex of each circular filament, where cells of stacked filaments are contiguous (i.e., adjacent). Most of the space between the filaments will be open and filled with cell culture media in vitro and interstitial fluid in vivo. Standard bioprinting devices are also unable to produce adjacent membranes and seed them with cells uniformly.

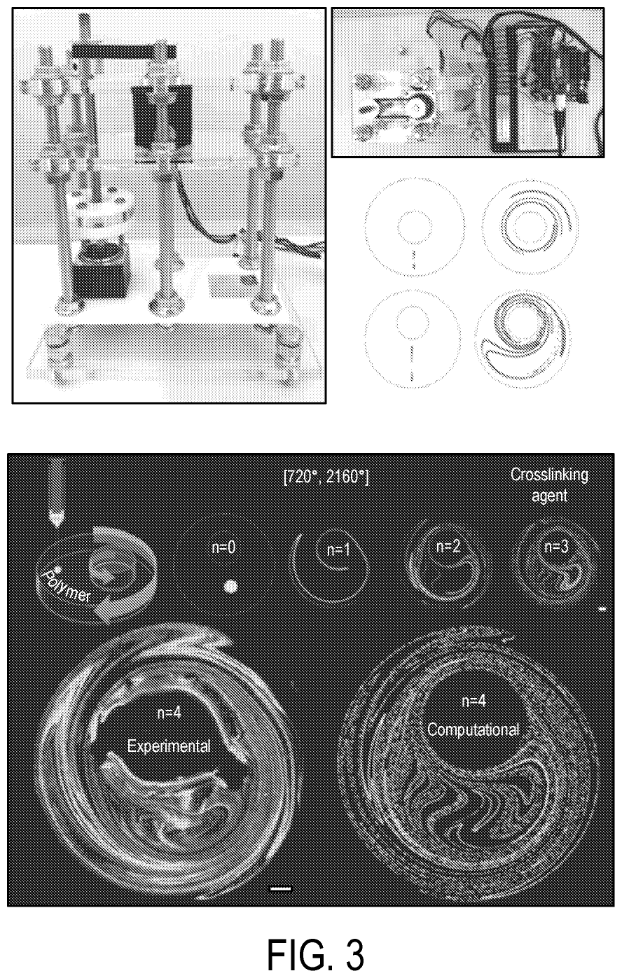

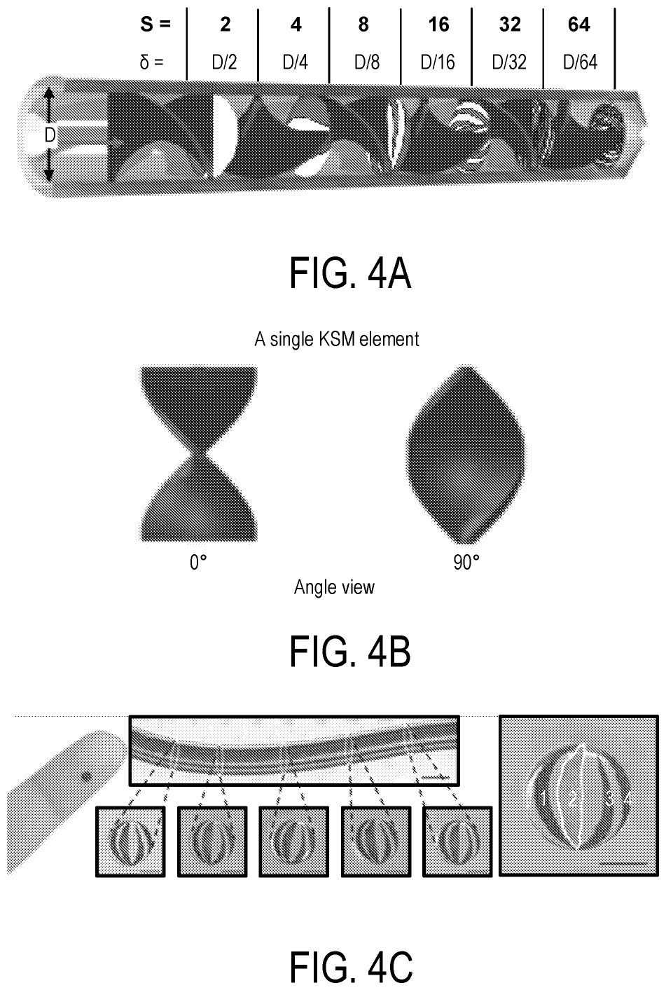

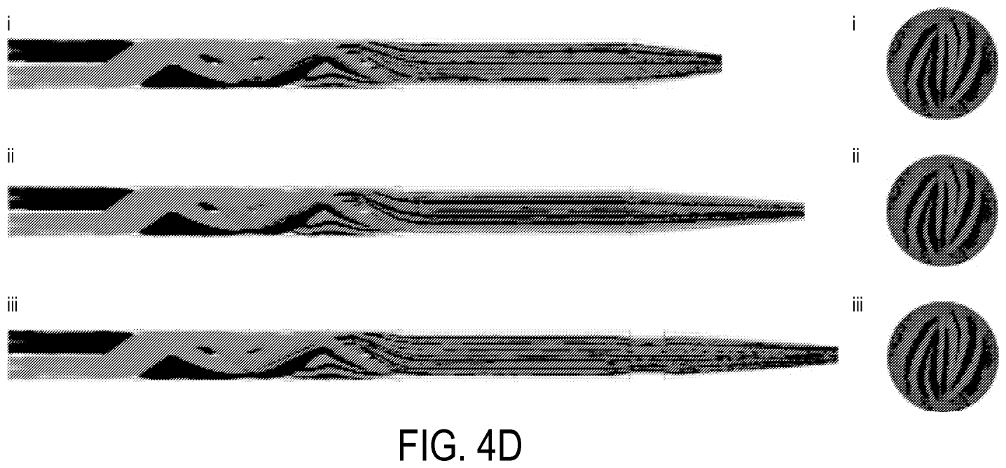

The concept of chaotic printing began with the observation, and use, of chaotic flows to create useful microstructures. Chaotic flows can be produced by turning the input cylinder of a “journal bearing” (JB) paint mixer in the opposite direction of the wall of the cylindrical tank within which are the correct ratios of different “mixing structures”. Likewise, a miniaturized version of the original JB, a miniJB (FIG. 3) can be used to fabricate multilayered microstructured constructs via a process termed “chaotic printing.” When the inner paint input rod and the outer mixing chamber wall turn in opposite directions, chaotic flows produce long lamina. These lamina shear alongside, rather than interpenetrate and mix with, each other as long as rheological conditions allow. These laminae can just as easily be fabricated with cell-laden hydrogels rather than different colors of paint. Further, chaotic layers can be formed by coextruding two polymers through a Kenics Static Mixer (KSM). The KSM (FIGS. 4A-4D) flows each input channel along a blade, a KSM element (FIG. 4B), causing the bioink to rotate 90 degrees where it then hits the edge of the next KSM blade (i.e., a second KSM element), thereby splitting the original two layers into four. If it is then flowed into and split by another KSM blade. There will now be eight layers, alternating between the original two types of cell-laden bioink, in a series of fully adjacent layers. In the correct rheological conditions, KSM chaotic printing extrusion nozzles can be extremely long without damaging the cells or resulting in unwanted mixing of adjacent layers (FIG. 4D).

Chaotic printing can also produce adjacent fugitive bioink layers for subsequent flow of cell culture media, in vitro cell seeding, in vivo cell homing, and/or in vivo vascularization. In this example, chaotically-printed layers containing fugitive bioink will be seeded with endothelial cells once that bioink evacuates and produces hollow channels. This is especially important in thick tissues as any highly-metabolic tissue must have all cells within 200 μm of: media in static culture, media flowing through a bioreactor in vitro, or blood in a vascular channel or capillary bed in vivo. In this example, we will seed both Bone Marrow-derived human Mesenchymal Stem Cells (BM-hMSCs) and Human Umbilical Vein Endothelial Cells (HUVECs) in contiguous chaotically-printed layers within filaments to produce bone ossicles. We will also compare printing layers of endothelial cells in sheets versus seeding endothelial cells in channels left by evacuated fugitive ink.

These initial studies are as much about establishing bone microvasculature and capillary beds as it is about bone tissue engineering. Despite more than three decades of research, there are currently no bone tissue engineering options for the surgical repair of critical size defects (CSD; 2.5 cm) or larger (i.e., defects that do not heal unaided). The standard-of-care for defects over 2.5 cm remains autologous bone graft. Bone grafts lead to pain and morbidity (i.e., die-back) at the donor site. Due to the aforementioned problems with vascularization, bone grafts are at risk of resorption or infection. Both of those undesirable outcomes occur in 5-10% of patients, depending on the anatomical site and the patient's overall health. The most promising technologies for tissue-engineered bone, e.g., Medtronic's Infuse™, have commonly been observed to fail at healing critical size and larger defects due to lack of persistent vasculature. Infuse is a Type 1 collagen sponge infused with BMP-2 inside of an inert plastic (e.g., poly(etheretherketone)) or metal (e.g., Ti-6Al-4V) cage that is inserted between two vertebrae that are to be fused following discectomy. The goal of the INFUSE product is to stimulate locally-available skeletal stem cells to fill an intervertebral space with new bone. The INFUSE product is used in subcritical size skeletal defects where its systemic release of BMP-2 has been associated with a wide range of deleterious effects, including nerve damage and inflammation, heterotopic ossification, and an elevated risk of cancer. Also problematic is the observation that while BMP2 may stimulate short-term bone formation, it can also stimulate long-term bone resorption (i.e., a short-lived restoration of bone in a defect site).

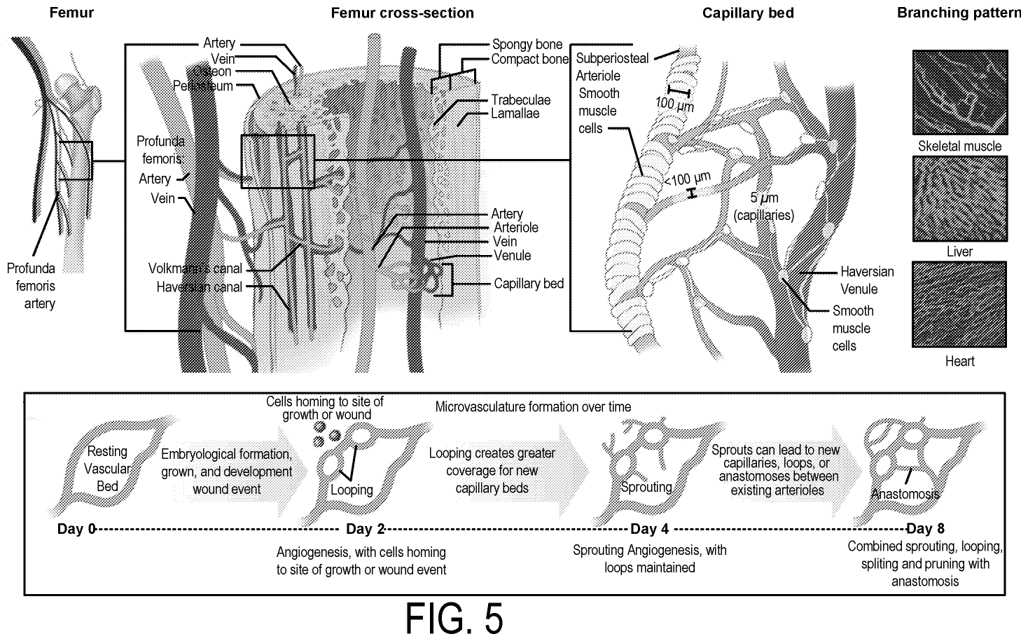

Autologous bone grafts, especially onlay grafts (i.e., no vascular pedicle), are usually placed near arteries, which in the case of the deep femoral artery are 2.5-7.5 mm in diameter (capillaries are 5 μm diameter). However, there is no guarantee that the microvasculature needed to connect these arteries to the newly placed bone will develop. It is expected that the de novo generation of graft-infusing microvasculature will take at least a week (FIG. 5), with full vascularization likely taking two weeks. At that rate, graft cell death will outpace vasculogenesis. Near the metaphysis and osteochondral junction of joints, specialized arterioles form by “bulging” rather than “sprouting” resulting in “H” arteriole architecture. One of the added constraints is that many researchers attempt to make their implants as strong as the original bone that was in the defect to be repaired. Very strong scaffold materials may prevent bone remodeling by acting as a barrier to the establishment of a microvasculature. It is important to note that, initially, chaotically printed devices would not be expected to be load-bearing, but rather would rely on fixation and immobilization of the wound site to heal (as in the Aim 3 rat model) and develop vascular systems that could support subsequent remodeling in response to unrestricted, normal use and loading patterns.

The initial success of a regenerative graft, either autologous or tissue engineered, in a wound-healing environment will fail to persist unless a perfusing microvasculature connects the new tissue to nearby arteries. Failure is more likely as animals increase in size. This may explain why successes in small animal skeletal repair models do not translate to humans. The methods described in this example will impact both the regenerative biofabrication of bone as well as the process of its microvascularization. Moreover, chaotic printing will be used to compare strategies of printing endothelial cells in layers, where sprouts and vessels are anticipated during in vitro culture, versus the post-printing seeding of endothelial cells in channels that open as fugitive bioink evacuates. In addition, the in vivo remodeling/parsing and persistence (i.e., evolution) of microvascular vessels (FIG. 5) for both autologous bone grafts as well as tissue-engineered bone grafts will be studied. This comparison of the two sources of bone is expected to bring the bone tissue engineering community closer to its longstanding sine qua non goal of creating a clinically viable source of “artificial” bone.

Current bioprinting and bioassembly, the two most common biofabrication techniques, are not capable of producing two thin membranes (sheets) of different types of cells in complete adjacency. Filament-based bioprinting (i.e., syringe-based extrusion) of HUVEC and BM-hMSC cell-laden hydrogels into a preformed solid scaffold has been achieved. Bioassembly (i.e., syringe-based extrusion of spheroids or tissue fragments into fugitive gel block, e.g., FRESH28 printing) of HUVECs/BM-hMSCs precultured spheroids into a hydrogel (fibrin) block housed within a solid scaffold has also been achieved. Neither of these techniques creates segregated layers of cells or prevents free mixing of different types of cells. The purpose of both of these studies was to create a sprout-laded construct, a prevascularized tissue engineered bone graft, that is hypothesized to have improved chances of establishing a microvasculature over constructs without endothelial cells and sprouts.

Two levels of innovation will be achieved in the proposed example. The first level of innovation would be the chaotic printing of these two types of cells in completely adjacent, contiguous layers within filaments. In the case of this example, we would use the original chaotic printing filament strategy to 3D-print a bone ossicle. Chaotic printing can place extremely thin layers of cells in adjacency within filaments without limit to the length of those filaments (FIG. 4D). This allows the second level of innovation applying chaotic lamina technology to sheet printing, thereby forming adjacent layers with complex shapes (e.g., curving surfaces). Thus, in this example, we extend filament chaotic printing to the production of flat and three dimensionally curving, shape-matching, and perfusing microvasculature sheets. Both the filament and sheet printing strategies would work with other types of stem cells. Moreover, these structures can be produced much faster than other methods of biofabrication that as resolution increases, biofabrication speed dramatically drops.

Co-culturing of contiguous sheets of HUVECs and MSCs will allow in vitro differentiation and simultaneous maturation towards vascular and load-bearing functional competence prior to implantation. In a SCID mouse model, co-culturing of HUVECs and BM-hMSCs can bring about HUVEC sprouting prior to implantation. However, this is not the normal order of events in wound healing. Microvasculature formation begins at the artery and progresses into the wound bed in response to hypoxic areas of healing and/or remodeling tissue. Remodeling in bone is usually a response to increased strain of aggregated osteoblasts or more mature osteocytes. The proposed creation of an adjacent, well organized (i.e., hierarchically branching) microvasculature is novel and more in line with normal bone wound healing processes thought to be controlled directly by the HIF-1α (Hypoxia Induced Factor) and Hippo (i.e., Yap1/Taz) pathways and indirectly by SDF-1 (Stromal cell-derived factor).

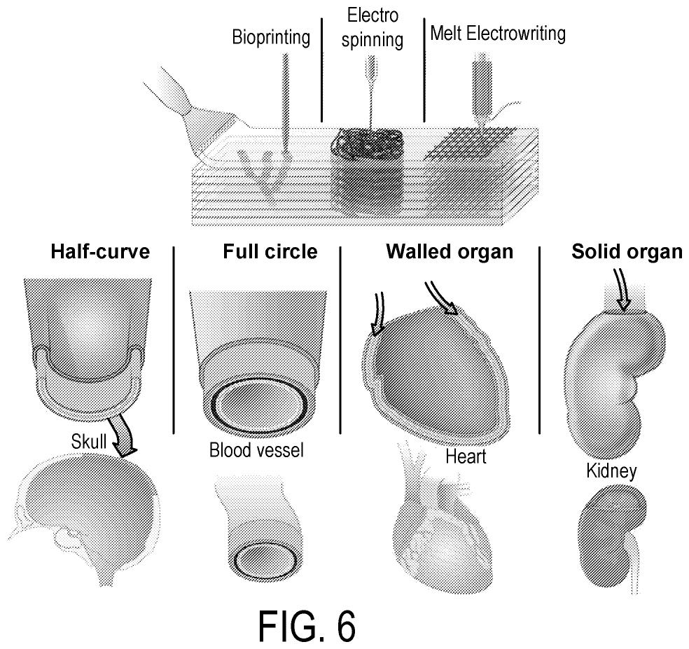

Extending chaotically printed sheets of endothelial cells (FIG. 6), that are connected to nearby host arteries during implantation, is a novel way of creating de novo microvasculature. Microvascular organ-specific morphology, in the case of bone, often owes to the fact that muscle is either covering much of a bone's surface and is the carrier of nearby arteries. This is also why grafted bone is often provided vascularized muscle coverage. Herein, we will also evaluate the evolution of chaotically printed microvascular sheets or post-print-seeded microvascular channels to see if the expected morphology is incipient. In addition to sprouting, we expect to observe “H” type endothelial cells, marked by high concentrations of CD-31/PECAM1 and EMCN [endomucin] content. As noted, these cells produce vertical “bulges” in the metaphysical and subperiosteal regions of cortical (strong) bone during postnatal growth and healing. “L” type bone endothelial cells predominate elsewhere in the diaphysis and are marked by low concentrations of CD-31/PECAM1 and EMCN (endomucin) content. A third type of endothelial cell, Type E, is found in bone primarily prenatally. It appears that Type H endothelial cells can actively direct the progression of bone healing (i.e., hematoma [blood clot], fibrous callus, and bone callus formation followed by bone shaping and remodeling). We will be tracking the relationship between microvasculature architecture formation and maturation and the presence of Type H endothelial cells. In this way, our work breaks with the current paradigm of “prevascularization.”

Chaotic Printing of sheet, tube, and solid devices will lead to future therapies that can bring about or restore complex organ function and allow for novel models of disease and therapeutic strategies. Anatomists see the body as a series of layers from superficial to deep, where some thicken, some involute, and others form channels due to physiological processes (e.g., blood flow, air intake, movement, digestion, lymphatic flow). Many embryologic, healing, and/or remodeling processes require layered tissue that tracks complex geometries such as convex surfaces (e.g., top of the skull), concentric layers within tubes (e.g., arteries), hollow organs (e.g., heart, bladder), and solid organs (e.g., kidney, liver, lung, spleen). FIG. 6 presents chaotic printing strategies to achieve the fabrication of these shapes. As layers are being lain down, other biofabrication modalities can be brought into play in directions orthogonal or parallel to the surface formed during chaotic printing. We have already combined chaotic printing and electrospinning. FIG. 6 also shows strategies for integrating syringe-based chaotic or bioprinting as well as melt electrowriting, a technology that can create extremely thin (10-micron range) polymer membranes that follow complex curving surfaces.

### Research Design and Experimental Methods

Specific Aim 1: Determine optimal cell density and/or layer thickness in adjacent, chaotic printed, bone marrow-derived human Mesenchymal Stem Cell (BM-hMSC) GelMA layers, Human Umbilical Vein Endothelial Cell (HUVEC) GelMA layers, and no-cell fugitive bioink layers to fabricate a vascularized, tissue engineered bone ossicle. We will also determine whether 3D printing HUVECs in hydrogel layers or seeding them in fugitive bioink channels is more effective.

Cell-bearing or fugitive hydrogel layers of BM-hMSCs and HUVECs can be chaotically printed in filaments in adjacent and uniformly dense layers. Post-print seeding of HUVEC channels will form a microvascular blood vessel geometry that performs better than a dense HUVEC layer.

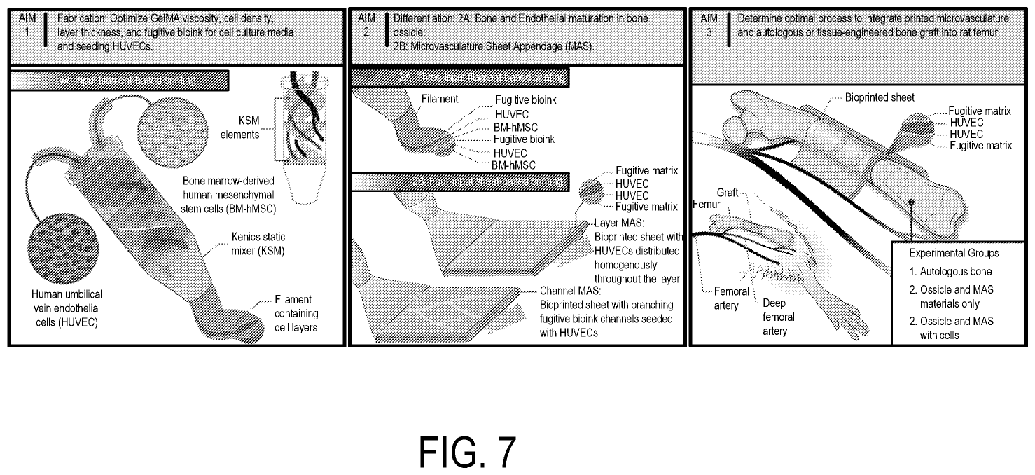

Aim 1 research is aimed, primarily, toward the first of the three primary goals of the three Specific Aims: Aim 1: Cell Seeding/Proliferation/Fugitive Channels; Aim 2: Cell and Tissue Differentiation/Maturation; Aim 3: Tissue Grafting/Healing/Restoring Physiologic Function (FIG. 7). In trying to seed cells, first in filaments, previously validated chaotic printing methods for creating layers of cells within filaments will be used to biofabricate a tissue-engineered bone ossicle that can then undergo Aim 1 cell proliferation and Aim 2 cell differentiation/maturation. More specifically, in the Aim 1 experiments we will produce contiguous layers of two types of cells, Bone Marrow-derived human Mesenchymal Stem Cells (BM-hMSCs) bone progenitor and Human Umbilical Vein Endothelial Cells (HUVECs) vascular progenitor cells. Once fabricated, these cell-laded filaments will be cultured in vitro and co-developed to attain organ-level function (i.e., artificial bone graft). Filament diameter, layer thickness, number of layers, and fugitive layer (i.e., after bioink removed) flow levels will be optimized to produce a bone ossicle that can be used in the Aim 3 rat femoral defect model.

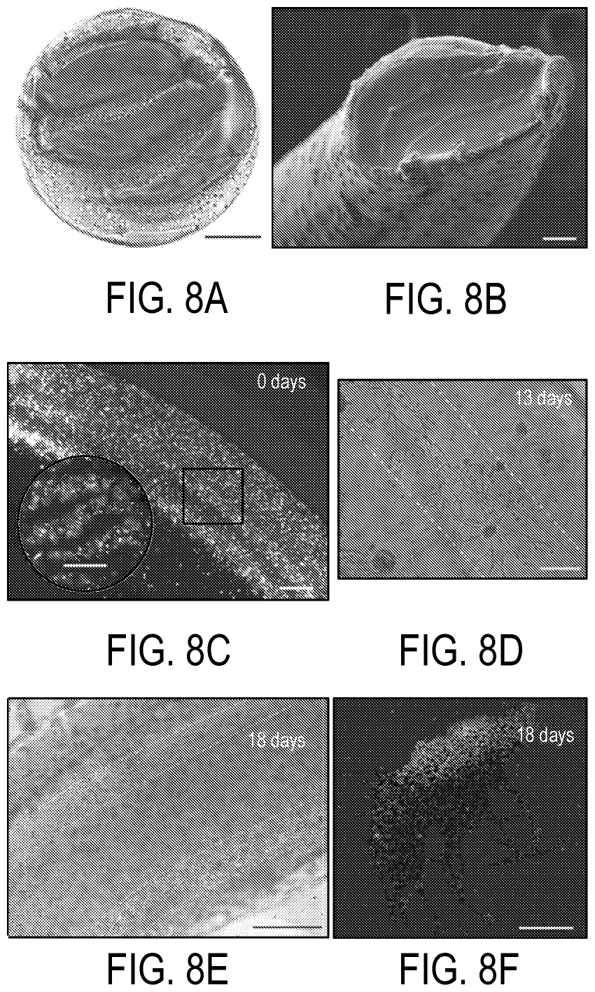

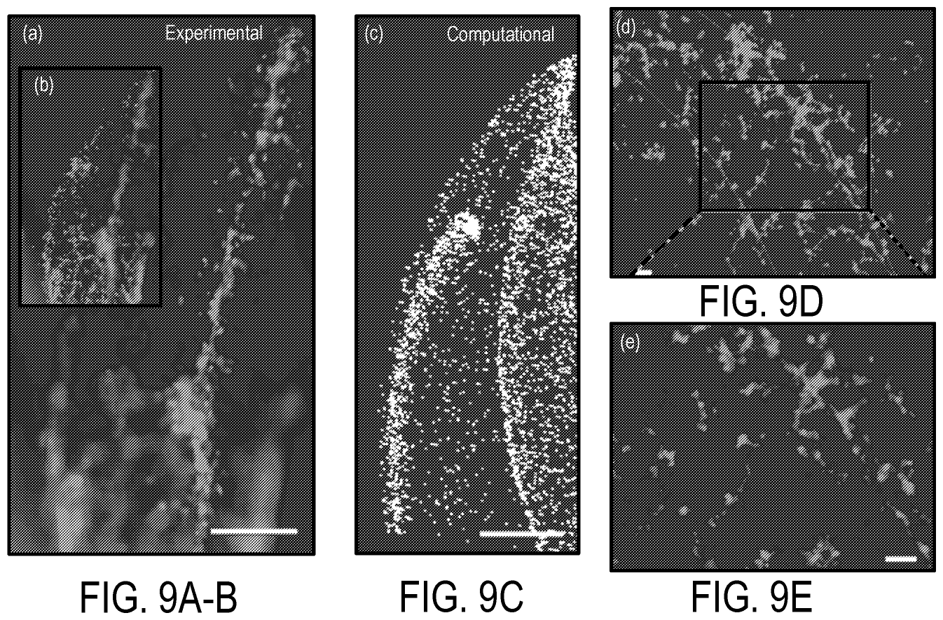

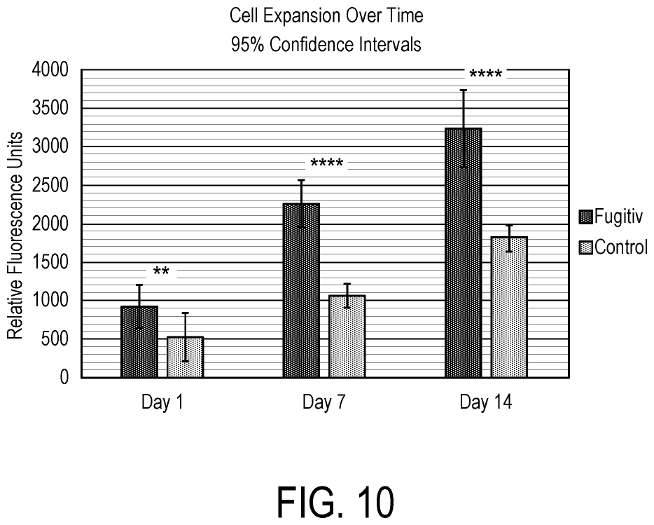

We have used chaotic printing to 3D print layers of muscle (C2C12) (FIGS. 8A-8F), immortalized breast cancer cells (MCF7), E. coli bacteria, endothelial cells (HUVEC) (FIG. 9), fibroblasts (L929), and mesenchymal stem cells (BM-hMSCs). Cell layers in these experiments have been routinely 50 m thick, which in cross-section is room for 3-5 cells, depending on cell type. In our published work we have used hydrogels rich in arginyl-glycyl-aspartic acid (RGD) motifs to maintain C2C12 cells within the layer in which they were printed next to layers without cells or RGDs.41 Our published work has also demonstrated that we can initially control the proliferation and spread of HUVEC cells by incorporating VEGF in HUVEC-bearing layers. However, within 3-5 days, adjacent cells of a different type, e.g., MCF7 cells (FIGS. 9A-9E), induced HUVEC in-migration. We have demonstrated the viability and proliferation of chaotically printed HUVECs in GelMA. We have also demonstrated the viability and proliferation of MSCs in GelMA (FIG. 10) as well as the utility of the fugitive bioink, hydroxyethyl cellulose (HEC). We have also optimized both cell proliferation and cell differentiation media for both HUVECs and BM-hMSCs. Those validated media will be used in this example.

As stated above, the overarching goal of the Aim 1 study is to determine optimal BM-hMSC and HUVEC cell seeding density and proliferation parameters for either a bone ossicle or a microvascular appendage sheet (MAS: see Aim 2). The initial study of cell seeding density (i.e., BM-hMSC and HUVEC), media components (to promote cell proliferation), chaotic layer number and thickness, hydrogel viscosity (i.e., depends on GelMA molecular weight), CaCl₂) crosslinker concentration, cell culture media fugitive layer parameters, RGDs and VEGF concentration (to promote cell localization/attachment) will be determined. One additional group will be optimized in addition to the standard chaotically printed filaments. This group will not have HUVECs printed in the vascular layer(s), but rather it will include fugitive bioink channels. Following biofabrication, the fugitive bioink will escape, with minimal intervention, opening up those channels for direct HUVEC seeding and cell culture media flow. Using starting parameters from our prior experiments, cell seeding density, channel thickness, and channel number will be optimized.

Cell-seeding will begin with optimization of GelMA stiffness for co-printing of BM-hMSC and HUVEC layers. The BM-hMSC and HUVEC layers will be loaded with RGD and VEGF, respectively. PrestoBlue (non-destructive) will be used to track cell proliferation. Confocal imaging and standard histology will be used to visualize the uniformity of cell distribution (see FIG. 9). Standard DAPI/GFP (MSC) and DAPI/RFP (HUVEC) fluorescent staining (destructive) will be conducted at 3-day intervals to confirm PrestoBlue data and to determine when to switch from proliferation to differentiation media. Osteogenic differentiation media will be tracked every 3 days with PrestoBlue, qualitative (DAPI, GFP/RFP) and quantitative (live/dead, alkaline phosphatase, and alizarin red S) microscopy, confocal imaging, IHC (Immunohistochemistry). Cell density and distribution quality will be assessed and scored (1=worst; 5=best). Scoring will be done by at least two “blinded evaluators.” The three best performing groups will be elevated to Aim 2 for further study. These four-layer chaotically printed filaments will have fugitive layers outside the two adjacent cell layers (i.e., BM-hMSC and HUVEC). Once these two fugitive layers clear, cell culture media optimized for proliferation (e.g., see FIG. 10) of the adjacent cells will be administered.

Using optimal strategies identified above, we will determine the most effective way to proliferate HUVECS in chaotically printed layers and in chaotically printed fugitive ink channels.

Experiments will optimize one parameter (e.g., cell layer loading density, viscosity, RGD/VEGF concentration, layer vs. channel seeding of HUVECs, etc.) at a time. The Aim 1 hypothesis suggests the following outcomes would be positive (desired). Positive outcomes are listed here in parentheses for each class of parameters: biofabrication materials and process (GelMA, alginate, and HEC viscosity can be similar enough to allow for adjacent layer printing; hydrogel stiffness allows cell survival), cell seeding success (successful attachment often determines survival), proliferation degree and distribution (are layers fully populated with sufficiently even thickness?), maintaining cell stemness during proliferation (there should be no mineralized bone extracellular matrix; other matrix constituents and genetic profile will be analyzed at the start of Aim 2 via RT-qPCR as secondary validation that this is the case) and the BM-hMSCs and HUVECs should retain high stemness.

The primary objectives for Aim 1 are: (a) to find the optimal biofabrication and cell proliferation strategies (cell density and/or layer thickness setting in chaotically printed fugitive bioink layers) and (b) to generate “good structure” (i.e., even and high distribution of the proliferated cells). The quality of the “structure” will be scored based on cell seeding efficiency and cell distribution (i.e., even density) where 5 is the best quality and 1 is the least desirable. We will generate at least five samples for each fabrication strategy and identify at least 3 fabrication strategies that yield a structure score of 4 or 5. Additionally, 4 samples will be generated from each of the selected fabrication strategies. One fabrication strategy will be used to validate the structure quality (i.e., high precision), and the remaining three will be elevated to the Aim 2 study. We will use “selection of the best” methods for determining overall sample size in our study.43 For these experiments, we will select one or more treatments that yields the best scores (highest or lowest mean values) among t groups to be used in the next experiment.

Our bioink materials (hydrogels) have been demonstrated to be compatible with the cells (i.e., BM-hMSC or HUVEC) in published studies or, in the case of BM-hMSCs, as presented here (FIG. 10). However, there may be difficulties matching the hydrogel stiffness (i.e., tuning viscosity) to be similar enough to allow adjacent layer printing (i.e., versus interpenetration or bilayer flow differential). Additionally, hydrogel stiffness, cell seeding and attachment success, and proliferation degree (i.e., are layers fully populated with sufficient even thickness) will determine cell survival.

Specific Aim 2: Determine the optimal nutrient and growth factor dose to differentiate BM-hMSCs to osteoblasts and to facilitate HUVEC formation of vascular micro-architecture including capillary beds in tissue engineered bone grafts. We will also use chaotic sheet printing to generate a Microvascular Appendage Sheet (MAS) to use as an aid to establish microvascular links between autologous or tissue free flap bone grafts and a nearby arterial blood supply.

Well-seeded and well-proliferated chaotically printed BM-hMSCs and HUVECS optimized in Aim 1, with either HUVECs in layers or channels, can be used to engineer bone ossicles, capillaries, and microvasculature in vitro (i.e., prior to implantation). Chaotic layer printing of Microvascular Appendage Sheets (MAS) can use layer or channel technologies to produce functional microvasculature with branching (hierarchical) geometry.

Aim 1 focuses on bilayer chaotic printing biofabrication and bone (i.e., BM-hMSC) and blood vessel (HUVEC) stem cell proliferation in adjacent layers. In its first in vitro project on differentiation, Aim 2 will take the best three of those results to determine the optimal differentiation strategy to produce a bone ossicle/graft for the Aim 3 rat femoral bone defect model. In the second in vitro project, a new chaotic layer printing technology (FIG. 6) will be tested for the production of Microvascular Appendage Sheets (MAS). The best MAS strategy will show hierarchical splitting and vessel geometry well suited to provide adequate rates of bone perfusion quickly after an autologous or tissue engineered bone graft. As shown in FIG. 7, the cylindrical bone ossicle scaffold will be precultured with and without a HUVEC sheet appendage. Scaffolds that include a sheet of HUVECs will provide a vascular sheet to wrap around the deep femoral artery during implantation in the Aim 3 rat model. That endothelial sheet of cells will be produced via a new device for chaotic layer printing described herein. This device represents a modification of the Kenics Static Mixer design (FIG. 7).

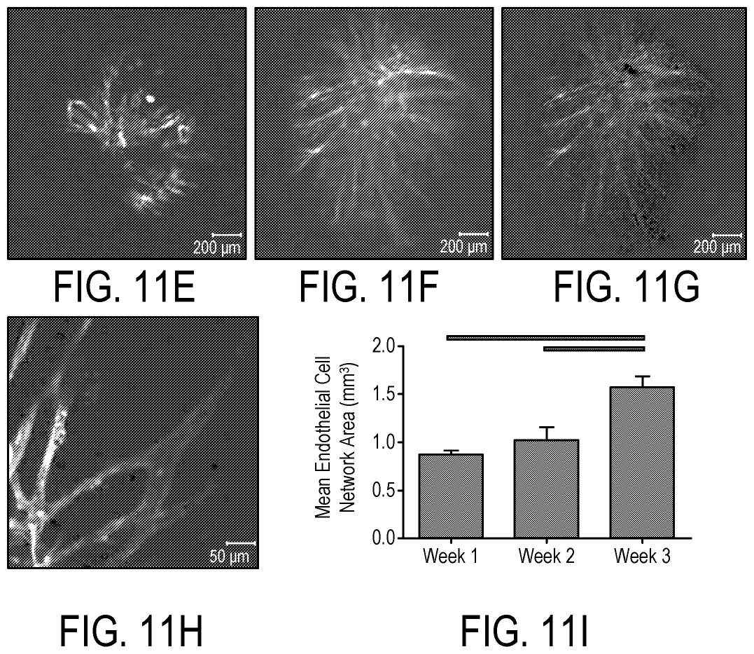

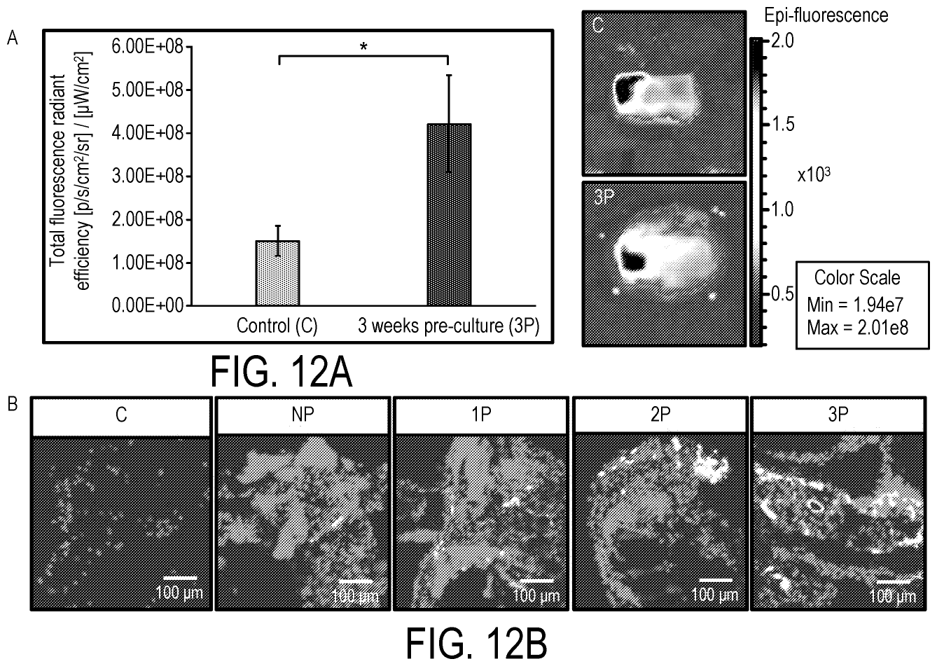

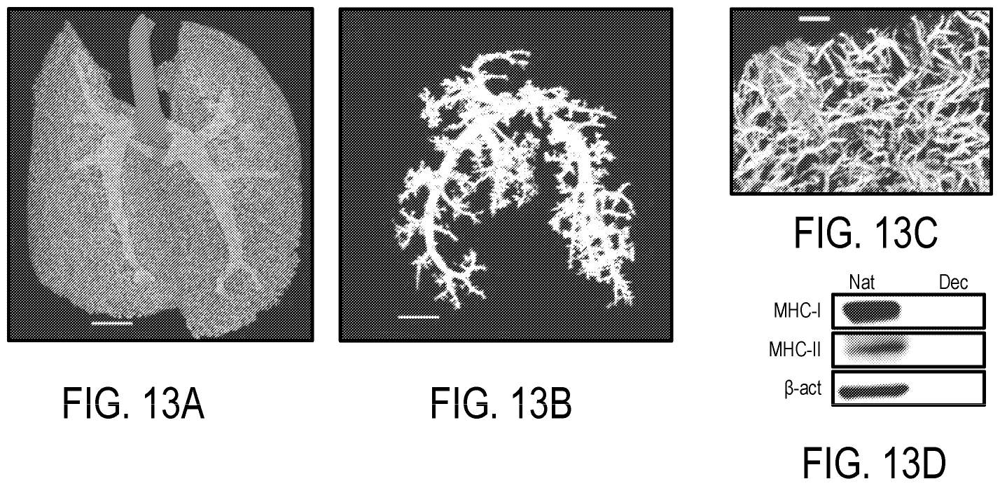

We have demonstrated the capacity to produce endothelial HUVEC microvascular sprouts from HUVEC and BM-hMSC spheroids bioassembled in fibrin scaffolds. These cells were co-cultured for 0, 1, 2, or 3 weeks. We observed more preculturing time resulted in a higher density of capillary sprouting (FIG. 11) and a more thoroughly perfused microvascular network (FIGS. 12A-12B) following implantation. Functional lung microvasculature (FIGS. 13A-13D) has been generated. Further, bone marrow mononuclear cells, which include mesenchymal stem cells (MSCs), can promote an inflammatory response that can facilitate a positive role of macrophages in the formation of small diameter to larger diameter vascular grafts. However, in this example, we will use only MSCs rather than an unsorted aliquot of bone mononuclear cells. Indeed, bone marrow-derived, allogeneic, including xenogeneic, MSCs are both immunomodulatory and are not detected by the host immune system unless they reach end stage (i.e., osteocyte). We will track the persistence and phenotype of these cells and other cells that home to the site of vascular and bone healing to make sure that we do not provoke an extended inflammatory response in the Aim 3 rat model.

These efforts will result in two structures. First, a chaotically printed filament, a microvasculature-infused bone ossicle (graft) derived from layers of both a BM-hMSCs and HUVECs. Second, a chaotically printed sheet will use HUVECs alone to produce a Microvascular Appendage Sheet (MAS). We will assess the quality of both bone and microvasculature in the former, and the microvasculature in the latter, during different in vitro culture intervals prior to implantation. The MAS is expected to perform in Aim 3 in a fashion similar to the current use of vascularized muscle or omental covering, now frequently used in the clinic and in tissue engineering research to provide quick, if not immediate, vascularization of autologous or tissue engineered implants.

The morphology of endothelial cells cultured in the three optimal Aim 1 layer-based HUVEC and channel-based HUVEC bone tissue engineering graft fabrication strategies will be studied at multiple time points (i.e., time 0, 1 week, 2 weeks, and 3 weeks). Osteogenic and vasculogenic media will be used. Imaging will be performed with osteogenic and vasculogenic cell differentiation markers. Capillary sprouting and the architecture (geometry of branches) of pre-microvascular will be imaged by overlaying CD-31 IHC and uCT images.

A new method will be studied in Aim 2. That strategy will be used to fabricate MAS sheets that promote HUVEC microvasculature capable of sustaining graft-promoting perfusion. The two MAS strategies will be biofabricated via chaotic printing with VEGF being used to localize HUVECS either during printing or later in the walls of fugitive bioink channels. The Microvascular Appendage Sheet (MAS) will be optimized to accept light suturing to hold it to either a bone graft or a tissue engineered bone scaffold. The effects of the thickness of layered HUVEC sheets versus the diameter (thickness) of branching, post-printing HUVEC-seeded channels will be optimized for use in the Aim 3 rat model.

These experiments will optimize bone quality and vascular perfusion. Positive outcomes are listed here for each class of parameters: quantitative Alkaline Phosphatase (conversion of MSC to osteoblast), Alizarin Red S (mineralization), uCT (mineralization), RT-qPCR and IHC bone phenotype. We will also optimize hierarchical branching from the arterial supply and perfusion of the graft. Positive outcomes are listed here in parentheses for each class of parameters: capillary sprouting (CD-31 confocal imaging), microvessel formation and hierarchical organization (uCT imaging), capillary and microvessel characterization via RT-qPCR and IHC.

It may be necessary to tweak the osteogenic or vasculogenic media to improve bone or vascular quality, respectively. As mentioned, we have experience optimizing both types of media. Problems with either could arise from the novel approach of chaotic printing compared to our earlier work utilizing both post-printing cell seeding or bioassembly. However, we expect that the major differences in materials or the higher resolution (i.e., thin membranes) will not be any more difficult to work with than the optimization needed in our prior studies that utilized chaotic printing.

Specific Aim 3: Determine the optimal chaotic printing process to integrate sheets of microvasculature and tissue engineered bone grafts in a rat femur model of bone regeneration.

A chaotically printed bone ossicle with a Microvascular Appendage Sheet (MAS) will result in rapid and full healing of a rat femoral diaphysis defect, irrespective of the defect-filling graft source (i.e., autograft or tissue engineered graft), and that has better mechanical properties than a graft without a MAS.

A rat femoral diaphysis defect model will be used to track microvascular perfusion of the bone wound (in vivo uCT scanning), its level of perfusion (Ultrasound), and quality of bone healing (ex vivo study of host integration and competency, IHC and RT-qPCR measures of healing maturity, i.e., cortical bone regeneration). Most importantly, the femoral diaphysis and deep femoral artery form a stable and predictable supply of blood for healing, bone formation, bone remodeling, and microvascular stability. Therefore, a competent microvasculature should be able to provide an early (i.e., within 3-4 days) and persistent blood supply. If successful, the vascularized bone ossicle (graft) resulting from the proposed study would have the potential to become the first “artificial” bone material for use in critical size and larger defects. Equally impactful would be a successful MAS. It would become a major adjunct to virtually all free flap tissue grafting, with the primary contributing factor being its reducing or eliminating the need to bring muscle, omentum, or other microvascular-rich tissues to a graft (e.g., autograft, tissue engineered) site. Finally, exploration of layer geometry, for the production of complexly-shaped bones or bone grafts for wound-specific defects, will also be a follow-on topic for chaotic printing, as will its translation to large animal models relevant to clinical musculoskeletal reconstructive surgery. Indeed, if the proposed project is successful, chaotic printing is well-positioned to provide physiological and biomechanical functioning layered tissue for tissue defects in an organ's curving surfaces, as well as tubular, hollow, or solid areas.

The Aim 3 rat femoral defect model involves a 5 mm diaphysis defect model that has recently been a frequent choice for assessing novel bone tissue engineering strategies and the success of microvascularization. That model will be used here to compare strategies for producing stable microvascular supply to bone grafts. The normal rat femur microvasculature will be compared to an autologous bone graft, with or without a chaotically printed Microvascular Appendage Sheet (MAS). Finally, the same comparisons will be made with chaotically-printed tissue-engineered bone grafts with or without a Microvascular Appendage Sheet.

Rat femoral defect model surgery and peri-operative tracking: Sixteen 4-month groups of 12 week old male and 12 week old female (50:50) (maximum N=10 animals per group). Altogether, this study will involve 96 rats total over 3 years. Thus, two 4-10 animal groups will be under study at any one time for 2.5 years with one week breaks between each 2-group sacrifice and the start of the next 2 groups. All 16 groups will be completed no less than 4 months before the end of the study. All animals will have a 5 mm (critical size) defect created mid-diaphysis with a 0.22 mm Gigli wire saw. All wounds will be fixated by standard rat femur fixation plates56 as far from the deep femoral artery as possible. Group 1 (control) will have the osteotomized bone removed and not replaced to confirm that spontaneous healing does not occur. Group 2 (control) will have the osteotomized bone reimplanted. Group 3 (control) will have the osteotomized bone reimplanted in the defect site with a chaotically-printed MAS with no cells or channels. Group 4 (control) will have the osteotomized bone reimplanted in the defect site with a chaotically-printed MAS with channels and no cells. Group 5 will have the osteotomized bone replaced with a chaotically-printed bone ossicle with chaotically-printed vascular layers with no HUVECs and no MAS. Group 6 will have the osteotomized bone replaced with a chaotically-printed ossicle with fugitive channels with no HUVECs and no MAS. Group 7 will the osteotomized bone replaced with a chaotically-printed ossicle with chaotically-printed vascular layers with HUVECs and no MAS. Group 8 will have the osteotomized bone replaced with a chaotically-printed ossicle with fugitive channels with HUVECs and no MAS. Group 9 will have a chaotically-printed ossicle with HUVECs in the original layer geometry placed in the defect site with a MAS that is chaotically-printed with layers and no HUVECs. Group 10 will have a chaotically-printed ossicle with HUVECs in the original layer geometry placed in the defect site with a MAS that is chaotically-printed with channels and no HUVECs. Group 11 will have an ossicle that is chaotically-printed with fugitive bioink in a channel geometry and subsequently seeded with HUVECS and a MAS that has chaotically-printed with the original layer geometry seeded with no HUVECs. Group 12 will have an ossicle that is chaotically-printed with fugitive bioink in a channel geometry and subsequently seeded with HUVECS and a MAS that has layer geometry with no HUVECs. Group 13 will have the osteotomized bone replaced with a chaotically-printed ossicle and chaotically-printed vascular layers with HUVECs and a MAS with layered HUVECs. Group 14 will have an ossicle that is chaotically-printed with an original layer geometry seeded with HUVECs and a MAS that has a channel geometry subsequently seeded with HUVECs. Group 15 will have the osteotomized bone replaced with a chaotically-printed ossicle with fugitive bioink layers that are subsequently seeded with HUVECs with a MAS that has chaotically-printed vascular layers seeded with HUVECs. Group 16 will have the osteotomized bone replaced with a chaotically-printed ossicle with fugitive bioink layers that are subsequently seeded with HUVECs with a MAS that has chaotically-printed channels that are subsequently seeded with HUVECs. These animals will be observed for 6-8 weeks57 and sacrificed. Groups with a MAS will have the MAS connected to the replaced bone or chaotically-printed bone ossicle and at the other end it will be sutured around the deep femoral artery. Prior to sacrifice, ultrasound58 and microCT vascular imaging will be conducted every 3 weeks (5 times pre-sacrifice).

Explant analysis: Post-operatively, the osteotomy sites will be analyzed by standard histology, IHC, and RT-qPCR for bone and vasculature per the Aim 2 regimen. Explants will also undergo compression, 4-point bending, and torsion biomechanical analysis. The deep femoral artery and surrounding microvasculature will be studied in terms of graft perfusion quality.

The overarching goal of this study is to test whether our chaotically-printed bone ossicle and MAS are useful in healing. Specifically, we will see if groups 13-16 yield better healing than groups 5-6. We will also evaluate the healing process in groups 13-16 vs group 2 (natural healing). Control 1, the negative control is a non-union (little to no healing expected). Control groups 3-6 will demonstrate that our hydrogel materials are biocompatible. In groups 7-12 we will study the effect of fabrication parameters (e.g., MAS with layers or channels) of these materials-only MAS treatments and will thoroughly vet the biocompatibility of the fabrication process.

### Example 2. Chaotic Printing of Microvascular Appendage Sheets (MASs) for Perfusing Tissue Engineered Constructs

The principal challenge to the clinical viability of large-scale tissue engineering is being able to perfuse entire constructs with blood supply that persists long-term. To accomplish this, tissue engineering must be able to replicate the hierarchically branching networks of microvasculature that distribute homogeneous blood supply from arteries to capillary beds in natural tissues. Chaotic printing is a bioprinting strategy that has been used to produce thin, cylindrical hydrogel fibers containing internal layers of cells at resolutions (˜10 microns) surpassing existing extrusion-based bioprinting devices (˜100 microns). We propose to utilize chaotic printing systems to extruding wide sheets of hydrogel containing hierarchically branching layers of multiple cell types that are native to vascular tissue. An in vitro “prevascularization” period can allow these cells to form into hollow tubular structures that mimic native microvasculature and can be perfused with blood supply. These prevascularized sheets, which we term “Microvascular Appendage Sheets” (MASs), can then be implanted in mice to demonstrate their ability to integrate with native arteries and veins and perfuse injured tissue with new blood supply. This example highlights the clinical potential of the MAS strategy and justifies its continued optimization in a subsequent large animal model.

### Overview

We hypothesize that chaotic printing can be adapted for engineering hierarchically branching “Microvascular Appendage Sheets” (MASs) that will promptly integrate with native vessels to provide complete, perpetual blood perfusion to specified tissues. The specific aims proposed herein are as follows:

Specific Aim 1: Design a system that can extrude hydrogel sheets containing hierarchically branching layer structures. The Chaotic Printing printhead currently used to extrude thin, cylindrical fibers of hydrogel will be modified to extrude wide sheets. The sheets will contain hierarchically branching layers of two hydrogel materials that can each be seeded with cells.

Specific Aim 2: Demonstrate the ability to bioprint Microvascular Appendage Sheets (MASs) and compare the effectiveness of three internal layer designs. This aim will verify that vascular cell types seeded into hydrogel sheets printed with the system from Aim 1 will organize into hierarchically branching vessel structures that can be sutured to vessels and perfused with blood.

Specific Aim 3: Verify functional integration of MASs with native vasculature in a mouse femoral defect model. MASs will be implanted in mice to determine whether successful integration with native vessels and perfusion occur in vivo.

Completing these aims will result in a system design that consistently and precisely produces hydrogel sheets with hierarchically branching internal layers. Furthermore, an in vitro “prevascularization” strategy will be established that results in cell-seeded MASs ready for in vivo implantation. Successful integration and functionalization in vivo will indicate the clinical potential of MASs and justify a large animal model to continue their optimization as a viable clinical strategy.

### Significance

Tissue perfusion is often recognized as central to the challenge of constructing large functional tissues or organs. While significant progress has been made, there remains a gap between lab-based engineered tissues and clinical translation. Autografting thus remains the clinical gold standard for tissue repair because flaps of soft tissue can be moved from one region to another while retaining their intrinsic microvasculature. It is important to first understand the general concepts behind common autografting procedures for the potential advantages of tissue engineering as an alternative to become realizable.

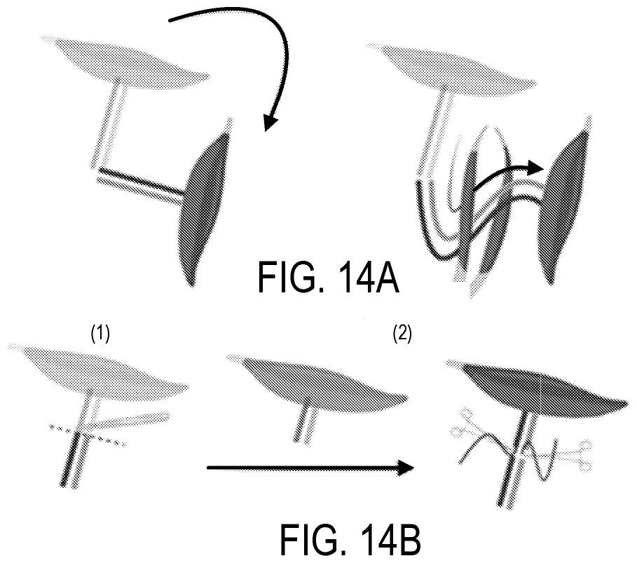

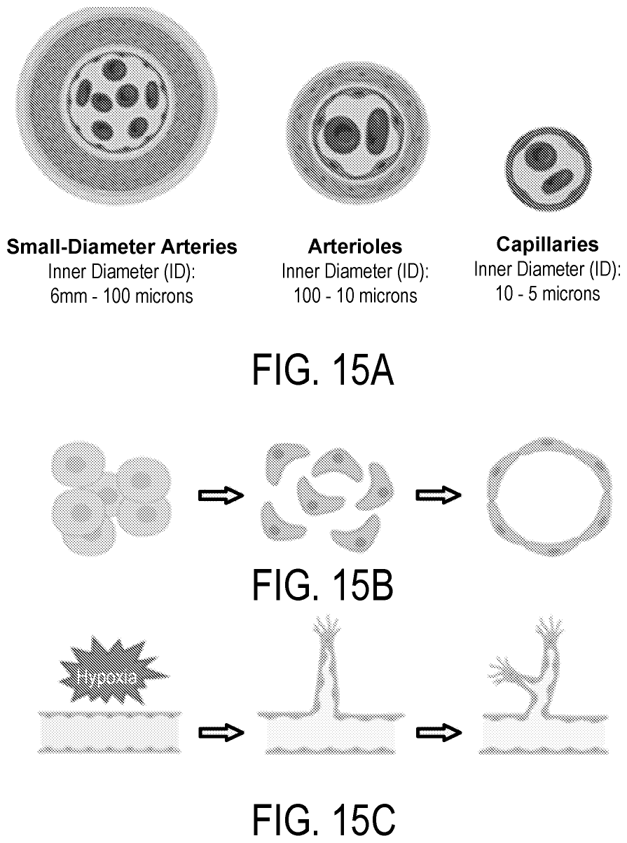

“Vascularized flaps” can be implanted both for the purpose of restoring tissue function directly or to promote perfusion as a supplement to wound healing. With “local flaps”, FIG. 14A, sections of tissue can be moved (i.e., rotated or tunneled) across a region with their supplying arteries and veins preserved. While this strategy preserves blood supply to the grafted tissue, there are a limited number of procedures where autologous flaps can be chosen from tissue within the same small region. “Free flaps” (FIG. 14B) are instead excised from their supplying arteries and veins while leaving a “pedicle” (i.e., the remaining length of these supplying vessels extending from the tissue). This allows free flaps to be transported an unlimited distance across the body as the pedicle is microsurgically sutured to small-diameter (i.e., Inner Diameter (ID)<6 mm) vessels local to the implantation site, but it comes with the possibility for failed vascular reintegration. This is usually rare (complication rate depends on specific procedure) but always devastating when it occurs, and is often attributed to thrombosis caused by failure to maintain blood flow in the microvasculature or exiting vein. Furthermore, all forms of autografting come with limited flap customization, source scarcity, and the inextricable result of tissue damage to the region from which the flap is removed. These are the primary disadvantages that tissue engineered grafts could bypass. To do so, however, they must be suturable to native small-diameter vessels in the implantation site and quickly establish blood perfusion across the whole construct with equally or more effective long-term results compared to pedicled autografts.

Decades of tissue engineered vascularization research across the globe have resulted in promising in vitro and small animal model capabilities curbed by lingering limitations when translated to large-scale tissue and whole-organ repair. We have demonstrated spontaneous organization of endothelial cells into capillary-scale (i.e., ID approximately 5 to 10 microns) vascular networks, with morphologies ranging from immature sprout, strand, or cord-like structures to those forming well-developed lumen. These spontaneously organized capillary beds have been successfully integrated with native vasculature in mice and remained stable and functional long-term (i.e., at least one year) in vivo.

Though these studies demonstrate successful capillary bed development and the incredible malleability of endothelial cells, spontaneous organization is not a reliable method for supplying large-scale tissues and organs. Spontaneously formed capillary beds are often spaced more than 200 microns apart, which is the diffusion limit of oxygen and nutrients within a tissue. This means that not all of the engineered tissue can receive sufficient nutrient supply rapidly after implantation, as is required. Furthermore, a spontaneously formed capillary bed on its own has no clear locations for surgical connection and integration (i.e., anastomosis) with native small-diameter vessels, causing perfusion of the network to be delayed until angiogenesis can, if at all, bridge the gap between native small-diameter vessels and the bed. Essentially, missing from the equation is microvasculature's natural function to hierarchically branch from small-diameter vessel-scale to capillary-scale, as well as to direct branching capillaries to regions less than 200 microns apart. Generation of proper microvasculature cannot be expected to occur from undirected “vasculogenesis” (i.e., endothelial cell organization into lumen) and “angiogenesis” (i.e., new branches sprouting from existing lumen).

Thus, the research focus has shifted toward creating hierarchically branching networks that resemble the natural organization of a vascular tree. Many novel fabrication methods are being applied to this pursuit, such as electrospinning, laser drilling/etching, casting and molding of materials, multi-material 3D fiber deposition, and 3D bioprinting.

Cell-permissive hydrogels are a preferrable channel material over dense biomaterials as they are bioresorbable, mimic the native Extra-Cellular Matrix (ECM) environment, and provide flexibility for endothelial cells to remodel the initially patterned vascular network. Some common hydrogels chosen for vascular engineering include forms of collagen, fibrin, gelatin, alginate, and decellularized ECM. The polymer chemistry of hydrogels allows their mechanical and chemical properties to be tuned with high specificity. Mechanical properties of engineered microvasculature must be finely tuned to balance promotion of desired cell activity with surgical requirements and maintenance of appropriate burst pressure (i.e., the max fluid pressure the construct can handle before structural failure). Growth factors or bioactive ligands can also be integrated into the hydrogel and released with spatiotemporal control to mimic natural mechanisms of vessel formation. This is an important capability for engineering microvasculature, as simply providing a support structure for vasculogenesis and angiogenesis generally isn't enough to provide complete control over the resulting cellular organization.

Crosslinkable polymer hydrogels are what make 3D bioprinting possible. Liquid hydrogels mixed with living cells can be positioned with high specificity before induced crosslinking transforms the gel into a solid, cell-laden construct. Bioprinting allows increased customization and the possible advantage of fabricating networks with hydrogel materials containing homogenously distributed cells, rather than requiring the constructs to be seeded or perfused with cells post-fabrication. Of the multiple bioprinting platforms in existence today, extrusion-based bioprinting is the most common due to its accessibility, compatibility with higher-viscosity hydrogels, and fast printing times. However, it generally has the lowest resolution with a minimum feature size of over 100 microns. This makes the current capabilities of extrusion-based bioprinting less suitable for producing microvascular and capillary-like structures (i.e., microvasculature ID being approx. 10 to 100 microns, capillary ID being 5 to 10 microns). Light-assisted bioprinting techniques, such as stereolithography (SLA), can be useful for bioprinting microvasculature due to their exceptionally high resolution, with feature sizes less than 10 microns.

Projection-based SLA has produced hierarchical vascular networks patterned directly into cell-laden hydrogels which significantly improved vascularization and anastomosis when subcutaneously implanted in mice.

The primary challenge to SLA is its reliance on ultraviolet (UV) light as an energy source, as UV light can cause DNA damage-induced cell death and mutagenesis, which is linked to carcinogenesis. The photoinitiator molecules required to induce crosslinking can also be mutagenic themselves. To address these barriers, next-generation lithography methods are in the early stages of implementing near-UV and visible light crosslinking. However, significant further development is required to reduce the cytotoxicity and mutagenicity risk of light-assisted strategies to a level that is acceptable for clinical applications.

### Innovation

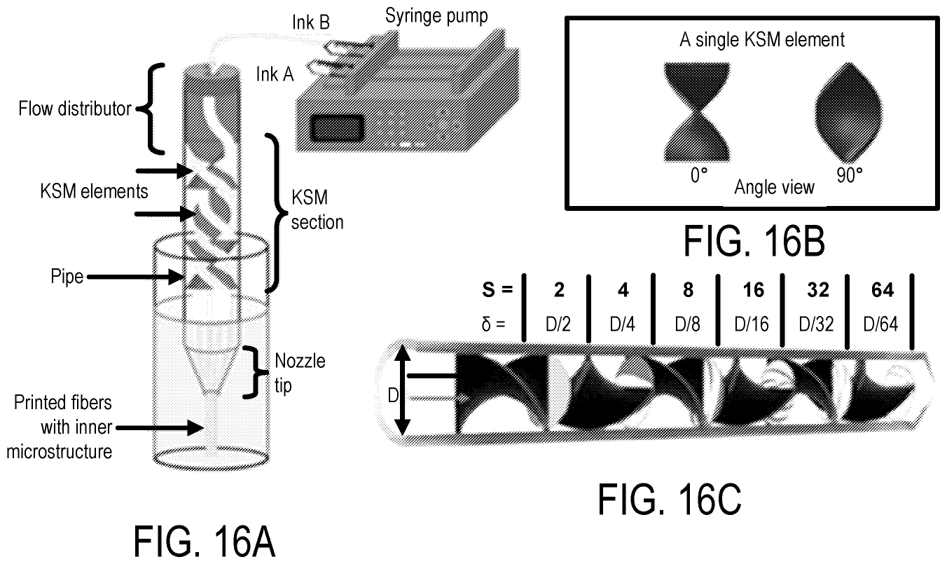

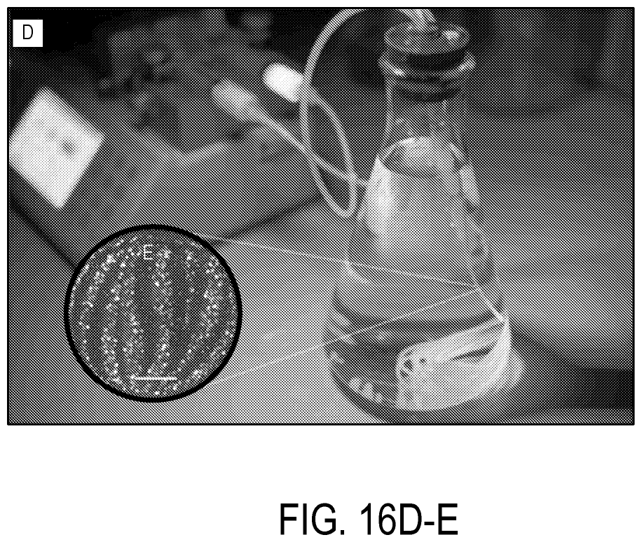

Chaotic Printing is a extrusion-based bioprinting technology with the potential to overcome multiple disadvantages of existing microvasculature fabrication strategies. Existing Chaotic Printing employs a printhead using the Kenics Static Mixer (KSM) design to successively bifurcate two hydrogel inputs into adjacent alternating layers within a single fiber. The number of layers within the fiber corresponds to the number of KSM mixing elements within the printhead (FIGS. 16A-16E). This provides precise control over the layer thickness extruded by the printhead up to a resolution significantly higher than existing state-of-the-art extrusion-based bioprinters (i.e., <10 microns versus >100 microns). Thus, Chaotic Printing could be the first extrusion-based microvascular fabrication strategy to bioprint at a resolution competing with light-assisted bioprinting without the associated concern for DNA damage.

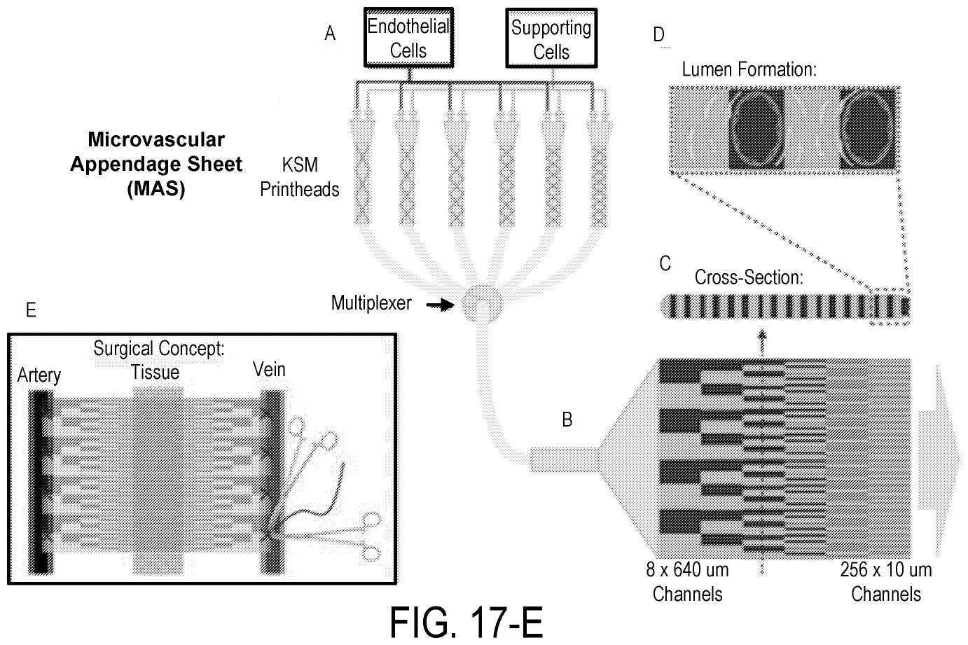

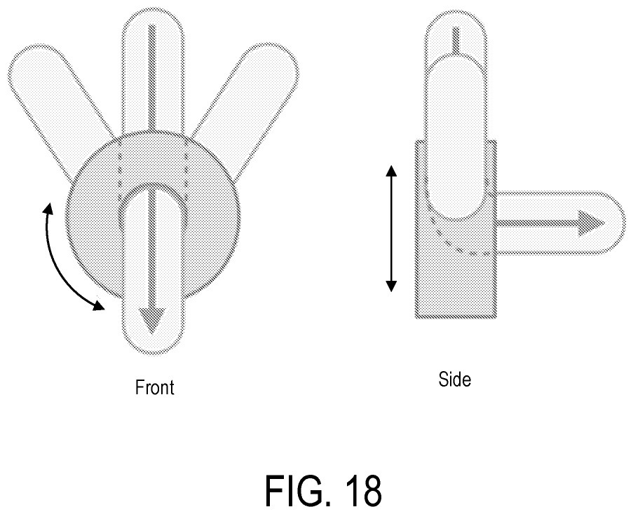

By using a “multiplexer” to vary the number of KSM elements the hydrogel inputs travel through during one extrusion (FIG. 18), a continuous gradient of decreasing layer thickness and increasing layer number can be fabricated within a single construct. While a few studies have managed to organize endothelial cells into hierarchically branching microvascular channels (i.e., one 300-micron channel branching to sixteen 25-micron channels, currently the smallest synthetic vessel generated within a cytocompatible biomaterial), no work to our knowledge has managed to produce a microvascular branching hierarchy in the range Chaotic Printing is easily capable of (i.e., eight 640-micron channels to two hundred and fifty six 10-micron channels) (FIG. 17). The ability to produce a microvascular branching hierarchy that spans millimeters to single microns could be the key to providing a continuous transition between capillary networks and native, microsurgically manipulatable small-diameter vessels (i.e., approximately 1 to 6 mm) for the first time in engineered tissues and organs.

New prototypes are also being developed to chaotically print wide sheets (i.e., length×width >>height) with internal layers. To our knowledge, extrusion-based bioprinting to this point has not been developed for printing wide sheets in one continuous extrusion. As one solid construct, these sheets would have improved structural integrity and faster fabrication times compared to sheets traditionally bioprinted with linearly deposited cylindrical fibers. In addition, their structure and dimensions could be beneficial for integrating grafts and native vessels via microsurgery (i.e., suturing one side to a small-diameter artery, wrapping around a construct, and suturing the opposite side to a small-diameter vein).

The adjacent internal layers produced by Chaotic Printing can include bioinks of two or more cell types, such as endothelial cells and supporting mural cells, either initially homogenized into the extruded hydrogels or later perfused into vacant layers left behind by a fugitive hydrogel. The sheet could also be coated in an additional cell-laden material to include three or more cell types. Pre-implantation, cellular organization into hierarchically branching microvascular networks could be promoted in vitro by integrating pro-vasculogenic and/or pro-angiogenic growth factors, such as PDGF and VEGF, and even stimulus-respondent release mechanisms allowing for spatiotemporal guidance of vessel formation.

If engineering microvasculature via Chaotic Printing proves to be as advantageous as predicted, it could be both developed into an independently viable clinical strategy as well as incorporated into other novel strategies for tissue and organ engineering and/or autografting that were not previously possible.

Specific Aim 1: Design a system that can extrude hydrogel sheets containing hierarchically branching layer structures. The system can incorporate one or more of: a fanning nozzle design that transitions from a cylindrical inlet to a wide outlet while conserving inner volume will transition the existing Chaotic Printing fiber printing process to producing wide hydrogel sheets with alternating internal layers; a multiplexer design that can quickly switch between hydrogel inputs mid-extrusion will consistently produce distinct delineations in layer number and thickness across the resulting sheet without jeopardizing its continuous, solid structure; and/or a “fugitive ink” prepared as one of the two hydrogel inputs to produce hierarchical branches of vacant internal layers within the resulting hydrogel sheet.

Aim 1 will be goal-based, rather than consisting of timed experiments. The primary goal is to come away with a system design that can consistently produce hierarchical layer branching within a 2-material hydrogel sheet. This will require producing 3D Computer Aided Design (CAD) and physical prototypes of a new Chaotic Printing combined printhead and fanning nozzle design (i.e., transitioning from tubular to wide while conserving inner volume), as well as a multiplexer design for modulating inputs from each printhead. This will likely require multiple design iterations to produce the desired results effectively and consistently as unexpected challenges emerge in translating the concept to a physical device. However, complete optimization will not be a focus of this design stage. Once its physical capabilities are verified through preliminary tests, the design will be used to print MAS seeded with appropriate cell types for producing functional microvascular tissue in Aim 2.

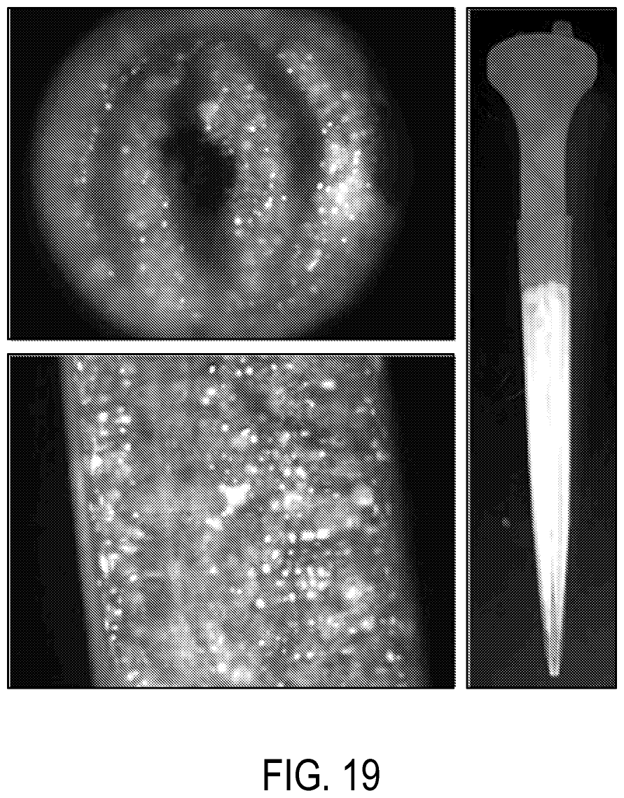

The foundations of the intended Chaotic system design for printing wide sheets have been established for printing thin, cylindrical fibers. For example, separate syringes containing each hydrogel input (i.e., 2% Sodium Alginate (SA) with and without green fluorescent particles) can be placed on a syringe pump and connected to KSM printheads via rubber tubing. With the syringe pump active, the two hydrogels mix into alternating layers and exit the nozzle at a set flow rate, which is critical for producing consistent results and keeping shear stress in the nozzle low enough to avoid cell death. The resulting construct can be extruded directly into calcium chloride (CaCl₂)) to induce SA crosslinking/solidification. The layers containing green fluorescent particles are illuminated when exposed to UV light, allowing the alternating layer structures to be observed (FIG. 19)

In addition, Hydroxyethyl Cellulose (HEC) has been used as a “fugitive ink” material. Instead of inputting two crosslinkable hydrogels, one is replaced with HEC. The resulting construct then contains alternating layers of crosslinkable hydrogel and HEC. Because HEC does not crosslink upon contact with CaCl₂), it remains liquid and evacuates the construct. Thus, the result is alternating vacant channels between solid hydrogel layers. This can increase cell proliferation rate by increasing contact area between cells and their nutrient media at the edges of each layer. With these protocols and design elements demonstrated, the design elements required to transition Chaotic Printing to producing wide hydrogel sheets are principally the fanning nozzle and multiplexer.

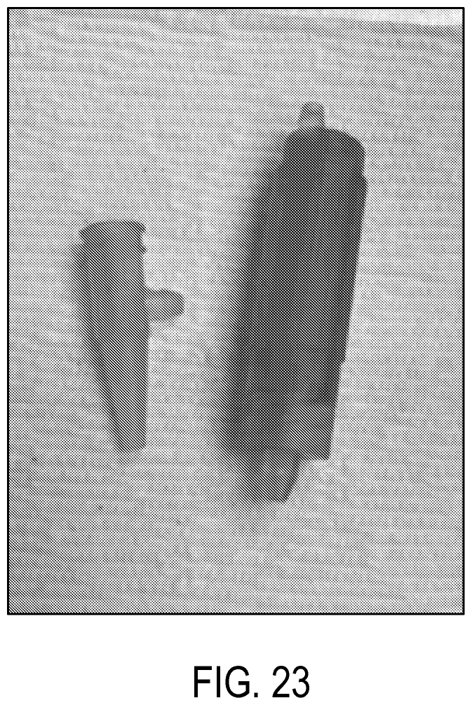

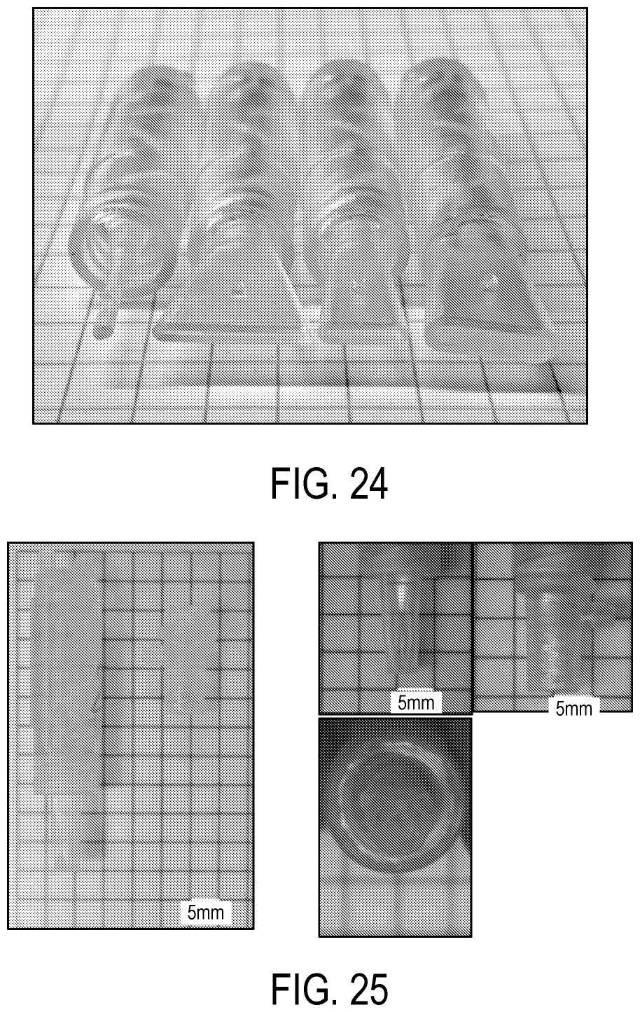

Proof-of-concept testing will begin with a preliminary printhead design that connects cylindrical KSM elements to a fanning nozzle outlet to demonstrate chaotic printing of hydrogel sheets for the first time (see, for example, FIGS. 23-25). The addition of a novel fanning nozzle design can be used to spread the cylindrical input from the KSM component into a wide sheet. This step will be considered successful when chaotically printed hydrogel sheets can be transported to a glass slide and alternating internal layers of green fluorescent particles can be imaged from a microscope supplied with UV light. Additional insights from this step may include determining geometric, flow rate, and SA weight/volume % limitations in producing the intended hydrogel sheet.

Once Chaotic Printing of an internally layered hydrogel sheet has been demonstrated, the next step will be to design a system for producing hierarchical branching of layers within the resulting hydrogel sheet. The key design challenge of this step is creating a multiplexer that can switch the final combined hydrogel output between multiple inputs from varying numbers of KSM sequential elements. The multiplexer must allow for a smooth shift between inputs so that the resulting hydrogel sheet contains continuous transitions between each layer hierarchy. This step can involve coding a switch mechanism in the multiplexer that could be controlled by a microcontroller from a company such as Arduino. This step will also conclude with visualizing the resulting layer structures microscopically under UV light, this time looking for hierarchical branching.

The same process for producing hydrogel sheets with hierarchically branching internal layers will then be attempted with one input being the HEC fugitive ink that does not crosslink upon contact with CaCl₂). The intention is to see whether hydrogel sheets can be fabricated with hierarchically branching internal layers that are vacant of material. This will be tested as one of three internal designs in Aim 2.

If these three goals are met with consistency, the system design will be considered ready to produce MASs incorporated with cells in Aim 2

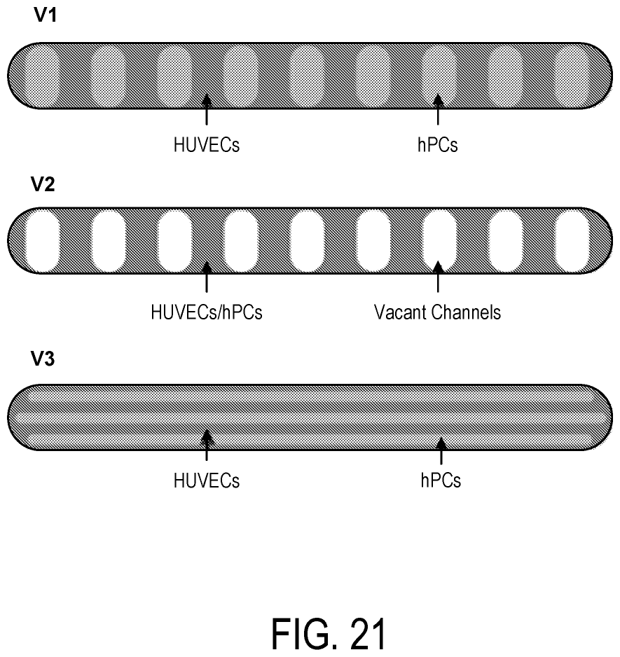

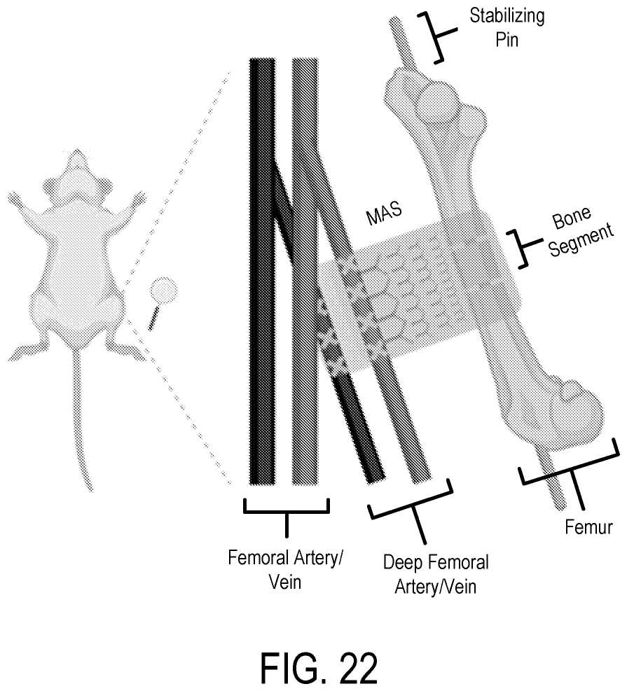

Specific Aim 2: Demonstrate the ability to bioprint Microvascular Appendage Sheets (MASs) and compare the effectiveness of three internal layer designs. This can include one or more of: a blended SA-GelMA polymer formulation will result in hydrogel networks that will allow cell proliferation and migration into hollow tubular structures (i.e., “lumen”) while being stiff enough to pass a suture retention test; a lumen will form in each MAS internal design (i.e., V1, V2, and V3; FIG. 21) that support continuous fluid flow from one end of the MAS to the other while maintaining a burst pressure greater than 500 mmHg; after 2 weeks of culture, MAS V1 will be determined to have the best overall performance in the each of the categories described above (i.e., cell migration/proliferation, suture retention, fluid flow, and burst pressure) and will be chosen for implantation in the Aim 3 mouse model.

Aim 2 will serve as in vitro proof-of-concept for the proposed MAS strategy, resulting in quantitative and qualitative data that justifies progression to in vivo experimentation. Cells will be incorporated into the system design developed in Aim 1 and three MAS internal layer designs will be compared. In vitro experimentation at this stage is expected not only to verify the potential for integrating MAS with host vasculature in vivo, but also indicate an effective in vitro prevascularization timeline of 2 weeks or less. The MAS design chosen in Aim 2 must be fully perfusable and suturable while maintaining an adequate burst pressure to be ready for in vivo implantation.

Cell Types. Numerous studies have demonstrated the pro-angiogenic effect of co-culturing endothelial cells and mural cells (i.e., pericytes). Because pericytes support, stabilize, and modulate newly-formed vessels, as well as remodel the vascular Extra-Cellular Matrix (ECM) environment, they are recommended to be included in bioink formulations when bioprinting microvascular structures. Thus, each of the two bioink inputs to our printhead will include human endothelial cells and human pericytes (hPCs), respectively. Pericytes are also known to guide angiogenesis by spatially restricting Vascular Endothelial Growth Factor (VEGF) signaling and creating paths to allow angiogenic invasion by endothelial cells. These traits should promote lumen development in the longitudinal direction within the hierarchically branching layers of the MAS.

There are additional pro-angiogenic supporting cell types which may be useful to include in later stages of optimizing the proposed strategy, such as Mesenchymal Stem Cells (MSCs), fibroblasts, and vascular Smooth Muscle Cells (vSMCs). However, a combination of endothelial and mural cells will be sufficient for a preliminary demonstration of the design's capabilities for producing vasculogenesis and angiogenesis. There are multiple endothelial cell types to choose from for microvascular tissue engineering as well. While microvascular endothelial cells are the most physiologically relevant, as their native function is to form capillaries in vivo, Human Umbilical Vein Endothelial Cells (HUVECs) are a popular cell type due to their high availability and robust expansion in culture. For the purposes of this project, HUVECs provide an economical option that can be utilized to demonstrate the potential of the system for inducing vasculogenesis and angiogenesis. Optimizing the system for specific applications will require careful consideration of the significant genotypic and phenotypic variations among microvascular endothelial cells depending on their location in the body, but this is beyond the scope of the proposed project.

Signaling Environment. Vascular morphogenesis (i.e., vasculogenesis and angiogenesis) and homeostasis in the body are regulated by biomolecular cues, both from paracrine and cell-ECM signaling. Blood vessels are surrounded by a dense basement membrane primarily consisting of type IV collagen and laminin proteins that maintain blood vessel homeostasis. Sprouting cells secrete proteases to degrade this basement membrane during angiogenesis to facilitate their proliferation and migration into the interstitial ECM. The interstitial ECM contains glycoproteins, such as fibronectin, collagen, and laminin, that interact with cell surface integrins to support vessel formation. The interstitial membrane also contains proteoglycans and glycosaminoglycans (GAGs) that can bind to angiogenic growth factors, such as VEGF and Fibroblast Growth Factor (FGF), and provide precise spatiotemporal control over their release for vessel patterning.

Example growth factors involved in vasculogenesis and angiogenesis in the body are listed below.

| | Function | |
|---|---|---|
| Growth Factor | In Vasculogenesis | In Angiogenesis |
| FGF | Cell migration, | EC migration |
| | proliferation, and | |
| | differentiation | |
| VEGF | Cell migration, | Vasopermeability, |
| | proliferation, and | Endothelial Cell (EC) |
| | differentiation, tube | proliferation, migration, |
| | formation | tubing and branching |
| PDGF | Mesenchymal Stem Cell | Pericyte recruitment |
| | (MSC) recruitment | |
| Ang1 | MSC-EC interaction, ECM | EC migration, tubing and |
| | deposition | branching, Pericyte-EC |
| | | junction formation |
| Ang2 | None | EC proliferation and |
| | | migration |
| TGF-β | MSC differentiation, EC | Regulating ECM deposition |
| | quiescence | |
| Proteinases (e.g. MMPs) | None | Regulation of ECM |
| and corresponding | | degradation, EC tubing and |
| inhibitors (e.g. TIMPs) | | branching |
| Nitrous Oxide (NO) | | Vasopermeability |

The bioinks used in this project need to be formulated with biomaterials that closely mimic the native vascular ECM environment. Blended hydrogels consisting of Sodium Alginate (SA) and Gelatin Methacryloyl (GelMA) have been successfully used as bioinks for chaotically printed fibers in which MSCs can survive for at least 21 days, as well as for printing microvessels seeded with HUVECs. In these structures, HUVEC cells were remarkably able to migrate through solid hydrogel and form lumen. This is an important finding that suggests HUVECs could do the same in solid layers of SA-GelMA hydrogel within MASs. The SA and GelMA components mimic a collagen-rich ECM environment that cells can easily attach to and migrate/proliferate within. Furthermore, GelMA can be finely tuned to user-defined mechanical properties based on degree of methacrylation and gel concentration and can promote endothelial cell adhesion and microvascular network formulation due to its retained RGD sequences (i.e., adhesion-promoting amino acid sequences found in collagen). SA and GelMA can be crosslinked in a non-cytotoxic manner by calcium chloride and low-energy, short-duration UV light exposure (i.e., 30 seconds at 365 nm), respectively. Thus, the first attempts at culturing HUVECs and pericytes in chaotically printed layered constructs will use SA-GelMA bioinks. Some companies, such as CellInk (Boston, MA), have created vascular tissue-specific bioinks that could be tested as alternatives if necessary.

The MAS must be cultured in a media formulation that provides basic cell nutrients as well as pro-vascular growth factors. Multiple companies (e.g., Lonza [Bend, OR], ThermoFisher [Waltham, MA], and Sigma Aldrich [Burlington, MA]) have specialized media formulations and/or supplements for HUVECs and hPCs, respectively. A common strategy for co-culturing two cell types that can be used in this project is to provide a 50:50 combination of media formulated each respective cell type. The biomolecular signaling the cells receive will be a combination of growth factors in the surrounding matrix, media, and those secreted by neighboring cells (i.e., paracrine signaling).

Several growth factors induce and regulate vascular morphogenesis in the body. Vasculogenesis is mediated by FGF, VEGF, Platelet-Derived Growth Factor (PDGF), Angiopoietin-1 (Ang1), and Transforming Growth Factor Beta (TGF-β). Angiogenesis is mediated by VEGF, Ang1, Ang2, FGF, PDGF, and TGF-β. More information about these growth factors and their roles in vascular morphogenesis is provided above. It should be noted that this is not a fully comprehensive list of every biomolecule involved in vascular morphogenesis processes, but it contains some of the most important factors. In addition, a hypoxic environment is also a key stimulus for angiogenesis in the body (i.e., presence of Nitrous Oxide (NO)). To our knowledge, few studies have attempted to stimulate angiogenesis in vivo by culturing cells in a hypoxic environment. On the contrary, most in vitro microvascular engineering work is conducted in conventional incubator conditions with high oxygen concentrations. While outside the scope of this project, future optimization of the proposed design could include observing the angiogenic effects of culturing in hypoxic conditions.

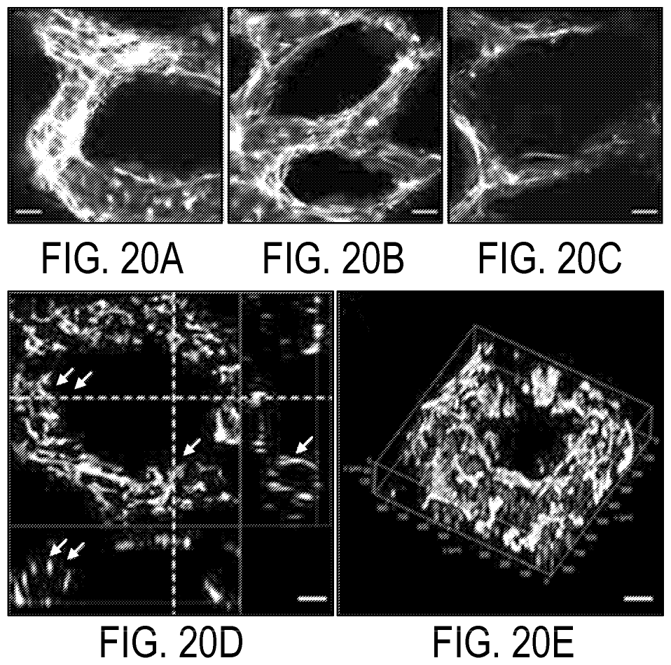

Histology Strategy. Fluorescence staining techniques can be used to image the morphological characteristics of HUVECs and hPCs within the MAS. CD-31 (green) for HUVECs and PDGFR-β (red) for hPCs have been used concurrently as fluorescent tags in previous hydrogel studies and should be effective in this project. The fluorescent imaging in this project can be conducted using a confocal microscope with z-stack function to reconstruct 3D views of the cell morphology within each MAS (FIGS. 20A-20E).

Tethered Ligands. Conjugating bioactive molecules to microvascular scaffolds can help to more closely mimic the complex physiological environment of native vessel formation. A strategy is currently being developed to synthesize short peptide sequences (i.e., “ligands”) of relevant growth factor active sites and tether them to the surface of biomaterial scaffolds. The benefits of growth factor-based therapies often come at the cost of adverse off-target effects resulting from freely circulating growth factors introduced to the body in unnaturally large doses. By comparison, tethered ligands could provide the benefits of relevant growth factors from molecules that are chemically bound to the scaffold. If ligands or ligand fragments break off the scaffold and enter circulation, they can only have the effect of the single active site rather than a whole protein. The ligands are synthesized with a catechol group so that they can easily bind to bioglass. Modulating bioglass concentration on biomaterial scaffolds allows for control over the ligand surface concentration. Tethering osteogenic ligands (i.e., Osteogenic Growth Peptide [OGP], basic Fibroblast Growth Factor [bFGF], and Bone Morphogenic Protein 2 [BMP2]) to bioglass-incorporated poly(propylene fumarate) (PPF) scaffolds improved human Mesenchymal Stem Cell (hMSC) adhesion, spreading, proliferation, and osteogenic differentiation at both gene and protein levels. A subsequent tethered ligands study has been planned to observe for the first time the effects of tethering OGP, bFGF, BMP2, and RGD in combination (i.e., all four ligands tethered to each individual PPF scaffold). This could provide valuable insights on how the tethered ligands strategy can be translated to engineering microvasculature in the future. Composite bioglass/SA and bioglass/GelMA hydrogels have been synthesized in other studies and could provide the means for tethering pro-vascular ligands to chaotically printed microvascular structures.

Aim 2 will take the system designed in Aim 1 and produce cell-laden MASs that can be used to collect data in a 2-week cell experiment. The primary goal is to consistently produce prevascularized MASs that can be implanted in an in vivo mouse model. To accomplish this, the following requirements should be met: (1) Passing a suture retention test, (2) Formation of lumen that allow continuous fluid flow from one end of the MAS to the other, and (3) Maintenance of a burst pressure less than 500 mmHg within the lumen.

The stiffness of the resulting hydrogel sheet must allow cell migration and proliferation through the polymer network as well as handling and suturing in microsurgery. This may present a challenge of conflicting needs, as a stiffer hydrogel matrix that may be ideal for surgical handling could also be too dense for cells to manipulate effectively. Verifying this step will require suture retention tests involving attempts to pierce the resulting MAS with sutures and add hanging weights to measure breaking strength and elongation, as described in detail and applied to hydrogels in previous studies. Suture retention tests should first be attempted with a cell-free hydrogel sheet before continuing to the 2-week cell experiment. However, since including cells later on will alter the stiffness of the hydrogel matrix, they should also be attempted again at the end of the cell experiment. If the suturing verification is unsuccessful or cells are not sufficiently motile in the 2-week cell experiment, hydrogel components and/or their weight/volume % will need to be revised. With continued suturing failures, a potential alternative strategy that could be explored is surrounding a softer cell-laden hydrogel sheet with a stiffer outer hydrogel coating that allows for surgical handling. In addition, an existing method of producing hydrogel-surgical mesh composites that can adhere to tissue could be applied to our project. These methods could also be implemented in further development even if suturing difficulties are not experienced in this project.

A short preliminary experiment can also be conducted on simple HUVEC and hPC-laden SA-GelMA constructs to observe whether cells can migrate, sprout, and form lumen within the SA-GelMA concentration determined to be suturable without cells. Adjustments to SA-GelMA concentration can be made at this point before conducting the more complex 2-week cell experiment.

The 2-week cell experiment will have experimental groups testing three inner layer designs: (V1) alternating, hierarchically branching layers of solid HUVEC-laden hydrogel and solid PC-laden hydrogel, (V2) alternating, hierarchically branching layers of solid HUVEC and hPC-laden hydrogel and fugitive ink (i.e., material that evacuates post-fabrication to leave vacant layers), and (V3) alternating wide layers of solid HUVEC-laden and solid hPC-laden hydrogel, without hierarchical branching (FIG. 21). The purpose of including these groups is to determine an optimal MAS internal layer design before moving to in vitro work. V1 and V2 are expected to have their own advantages and disadvantages. For example, V1 should result in stiffer MASs with better suture retention, and HUVECs and hPCs are initially divided into separate groups, but HUVECs must form lumen through an initially solid hydrogel network. While this has been shown to take place in other studies, V2 might result in quicker and/or more effective lumen formation as the HUVECs are already distributed around vacant channels. However, this could also have the opposite effect of limiting potential migration pathways of cells that could have otherwise traversed across the hydrogel network. V3 is effectively a control group, included to determine how much of a positive effect the hierarchical branching strategy has on directing vascular morphogenesis in the MAS. The V3 MASs will be more reminiscent of existing strategies that have laid sheets of two cell types on top of each other for the purpose of engineering microvasculature.24 Each group will have four replicates per data collection point. MASs in the 2-week experiment will be cultured in ultra-low-attachment well plates to promote preferential cell adherence to the hydrogel network over the well plate surface.

Data collection during the 2-week cell experiment will include conducting PrestoBlue (Invitrogen; Waltham, MA) metabolic assays and confocal 3D Z-stack fluorescent microscopy on days 0, 1, 7, and 14 post-printing. The purpose of using PrestoBlue is to ensure cell viability in the MAS across the experimental period and observe whether changes occur and to what extent. The resulting PrestoBlue fluorescence intensity data from a Cytation 5 Multi-Mode Reader (BioTek; Santa Clara, CA) will allow viability to be compared across each group over time via pairwise comparison statistical tests. The confocal Z-stacks will provide 3D renders of MASs from each group with fluorescent tagging of CD-31-tagged HUVECs and PDGFR-B-tagged hPCs. Cell morphology can then be observed, both quantitatively and through multiple quantitative measurements utilizing 3D distribution of fluorescence intensity (i.e., lumen inner diameter, lumen wall thickness, lumen density per area, PC layer thickness around lumen, cell type distribution).

Also on day 14, MASs will be connected to circulating fluid flow with an existing bioreactor design, with video recording of dyed water moving through the constructs as verification of complete liquid perfusion. With this same setup, burst pressure will be calculated by pressuring the MASs with fluid until failure, as described in previous studies. Burst pressure must be greater than or equal to 500 mmHg to be considered safe for connection to a mouse artery.

Because pericytes regulate lumen diameter and capillary blood flow through contraction, it is likely they will have some amount of shrinkage effect on the hydrogel matrix. Thus, MAS dimensions will be measured on day 14 of culture and compared to day 0 to determine a simple model for shrinkage compensation.

As mentioned previously, breaking strength and elongation from the suture retention tests will be collected with a cell-free construct as well as after the 2-week cell experiment.

For all statistical analysis, groups will be tested for normality and equal variance assumptions and a P-value <0.05 will be considered significant.

Specific Aim 3: Verify functional integration of MASs with native vasculature in a mouse femoral defect model. This can include one or more of: the MAS internal design (i.e., V1, V2, or V3) chosen from Aim 2 will integrate with the deep femoral artery and vein in the mouse hindlimb to be fully perfused with native blood supply, sustained for at least four weeks; the left hindlimb with an MAS wrapped around a femoral defect will have less necrotic damage and improved healing compared to the right hindlimb with a femoral defect but no implanted MAS (i.e., the negative control).

Aim 3 will take MASs formed with the determined optimal internal design and in vitro prevascularization methods from Aim 2 and implant them in mice for 4 weeks to observe whether they form a robust connection with native vessels. To provide proof-of-concept for the MAS's ability to bridge a blood supply gap between native vessels and specified tissues, the MAS will be sutured to the deep femoral artery, wrapped around a femoral defect, then sutured to the deep femoral vein. Histological samples will be analyzed to determine the morphology and expression of the HUVECs, hPCs, and bone tissue after four weeks post-implantation, and the presence of lectin and red blood cells will indicate whether blood perfused the microvascular network. The MAS has no clinical importance if it cannot integrate with native vessels, making Aim 3 a crucial demonstration. Ideally, the completion of Aim 3 will present the Chaotic Printing MAS strategy to the tissue engineering community in a position of clear clinical potential that demands further optimization and development toward specified applications.

Surgical Strategy. Arterial anatomy of the mouse hindlimb has been well characterized for the purpose of numerous ischemia models. In addition, there are multiple mouse models for creating femoral defects and studying subsequent bone healing. For the purposes of this study, a 3-mm segment will be created in the femur by cutting through the shaft in two locations 3 mm part. The segment will then be stabilized in place with a K wire (i.e., pin). A prevascularized MAS (i.e., having undergone vasculogenesis in vivo as in Aim 2) will be sutured to the deep femoral artery and wrapped around the surface (i.e., the periosteum) of the segmented femur shaft. [FIG. 9]. The opposite end of the MAS will then be sutured to the deep femoral vein to provide blood outflow. It is expected that the MAS will form a bridge between the origin of the deep femoral artery and the avascularized bone segment to promote healing by providing new blood supply. While this exact surgical procedure will be novel many general mouse surgery protocols for this project can be adapted from the existing mouse femoral defect models.

Severe Combined Immunodeficiency (SCID) mice have been used in previous models of integrating tissue engineered microvascular structures and will benefit this project by allowing continued use of human-derived cell lines from Aim 2 to 3.

Histology Strategy. In addition to the staining techniques used in Aim 2 to observe cell morphology, Aim 3 must determine whether each MAS has integrated with the deep femoral artery and begun to transport blood through its microvascular network to the femoral defect and back out to the deep femoral vein. This can be accomplished using fluorescent tomato lectin, a strategy utilized in previous experiments with tissue engineered microvascular constructs. The method involves injecting lectin into the mouse tail vein, which travels through the blood stream and binds to endothelial cells it comes in contact with. Thus, observing lectin fluorescence within the MAS network can indicate its perfusion with blood from the deep femoral artery. In addition, Hematoxylin & Eosin (H&E) staining has been used to identify red blood cells in tissue engineered microvasculature, another indication of blood perfusion, and can also be used to observe bone healing.

All procedures will be performed under an Institutional Animal Care and Use Committee (IACUC) approved protocol. The experimental group will consist of MASs with the internal layer design determined to be most efficient in Aim 2. A negative control will be included on the contralateral limb to observe the effect the MAS has on revascularizing the bone defect and promoting healing. Each group will contain 3 SCID mice per data collection point. In vitro prevascularization period length (i.e., in vitro culture of cell-laden MASs) will be based on results from Aim 2. After prevascularization, the MASs will be transported to the surgery room for implantation. Mice will be anesthetized, shaven, and sterilized before a longitudinal incision is made in the left thigh. The bone shaft will be visualized with an incision along the septal line between thigh muscles and a periosteal elevator. The 3 mm section will be isolated from the femur shaft using a diamond-coated rotary cutter tool with saline irrigation, then fixed in place with a K wire. The MAS will be sutured to the deep femoral artery and wrapped around the bone segment to be in contact with its periosteum, which will be carefully preserved. The opposite side of the MAS will be sutured to the deep femoral vein. As the negative control on the right thigh, the same bone segmentation procedure will be conducted without implanting a MAS. Local anesthetic will be provided during the implant procedure, as well as pain management and anti-inflammatory drugs post-surgery.

After four weeks post-implantation, the mice will be euthanized and fixed for paraffin embedding and histological analysis. As in Aim 2, cell morphology will be analyzed qualitatively and quantitatively by tagging tissue samples with CD-31 and PDGFR-β to identify HUVECs and hPCs, respectively. In addition, three randomly selected mice from each group will receive lectin staining via a tail injection to observe whether the MAS lumen were perfused with native blood supply. This will be quantified with 3D distribution of lectin fluorescence intensity and pairwise comparisons will be calculated for each group, as with the CD-31 and PDGFR-β immunohistochemistry. Three more mice from each group will receive H&E staining to identify red blood cells in the MASs, another indicator of blood perfusion, as well as to observe bone tissue healing and/or necrosis.

The compounds, compositions, and methods of the appended claims are not limited in scope by the specific compounds, compositions, and methods described herein, which are intended as illustrations of a few aspects of the claims. Any compounds, compositions, and methods that are functionally equivalent are intended to fall within the scope of the claims. Various modifications of the compounds, compositions, and methods in addition to those shown and described herein are intended to fall within the scope of the appended claims. Further, while only certain representative compounds, components, compositions, and method steps disclosed herein are specifically described, other combinations of the compounds, components, compositions, and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

Unless defined otherwise, all technical and scientific terms used herein have the same meanings as commonly understood by one of skill in the art to which the disclosed invention belongs. Publications cited herein and the materials for which they are cited are specifically incorporated by reference.

What is claimed is:

1. A method for preparing a non-filamentous scaffold for cell or tissue culture, the method comprising:
providing at least a first printing composition and a second printing composition;
chaotic printing the first printing composition and the second printing composition to generate a microstructured precursor comprising a plurality of lamellar structures formed from the first printing composition and the second printing composition;
extruding the microstructured precursor through a nozzle to produce a non-filamentous microstructured precursor; and
curing the non-filamentous microstructured precursor to provide the non-filamentous scaffold for cell or tissue culture.

2. The method of claim 1, wherein the nozzle comprises a fan-shaped nozzle.

3. The method of any of claims 1-2, wherein the non-filamentous microstructured precursor and the non-filamentous scaffold for cell or tissue culture comprise a sheet.

3. The method of claim 3, wherein the sheet has a width and a height, and wherein the width of the sheet is at least five times the height of the sheet, such as at least ten times the height of the sheet.

4. The method of claim 1, wherein the nozzle comprises a curved fan-shaped nozzle or annular nozzle.

5. The method of claim 4, wherein the non-filamentous microstructured precursor and the non-filamentous scaffold for cell or tissue culture comprise a curved sheet or hollow tube.

6. The method of any of claims 1-5, wherein the nozzle exhibits a substantially non-circular cross-section.

7. The method of any of claims 1-6, wherein the non-filamentous microstructured precursor and the non-filamentous scaffold exhibit a substantially non-circular cross-section perpendicular to an axis along which extrusion occurs.

8. The method of any of claims 1-7, wherein the first printing composition comprises a bioink composition

9. The method of any of claims 1-8, wherein the second printing composition comprises a bioink composition.

10. The method of any of claims 1-9, wherein the second printing composition comprises a fugitive ink composition.

11. The method of claim 10, wherein the method further comprises removing the fugitive ink composition from the non-filamentous scaffold following curing.

12. The method of any of claims 8-11, wherein the method further comprises dispersing a population of cells in the bioink composition prior to the chaotic printing.

13. The method of any of claims 1-12, wherein the method further comprises seeding the non-filamentous scaffold with a population of cells.

14. The method of any of claims 12-13, wherein the cells comprise pluripotent stem cells, multipotent stem cells, progenitor cells, terminally differentiated cells, endothelial cells, endothelial progenitor cells, immortalized cell lines, primary cells, or any combination thereof.

15. The method of any of claims 1-14, wherein chaotic printing of the first printing composition and the second printing composition comprises inducing laminar flow of the first printing composition and the second printing composition through a mixer that chaotically mixes the first printing composition and the second printing composition to form lamellar interfaces between the first printing composition and the second printing composition.

16. The method of any of claims 1-15, wherein chaotic printing of the first printing composition and the second printing composition comprises coextruding the first printing composition and the second printing composition through a mixer that chaotically mixes the first printing composition and the second printing composition to form lamellar interfaces between the first printing composition and the second printing composition.

17. The method of any of claims 15-16, wherein the mixer comprises a static mixer, such as a Kenics static mixer.

18. The method of any of claims 1-17, wherein the chaotic printing of the first printing composition and the second printing composition comprises coextruding the first printing composition and the second printing composition with a crosslinking agent.

19. The method of claim 18, wherein the first printing composition comprises an alginate and wherein the crosslinking agent comprises a divalent cation.

20. The method of claim 19, wherein the crosslinking agent comprises a calcium salt such as calcium chloride.

21. The method of any of claims 1-20, wherein the non-filamentous scaffold exhibits an average striation thickness of from 10 nm to 200 μm.

22. The method of any of claims 1-21, wherein the non-filamentous scaffold exhibits a surface-area-to-volume (SAV) of from 400 m⁻¹ to 5000 m⁻¹.

23. The method of any of claims 1-22, wherein the non-filamentous scaffold exhibits a surface density of at least 0.05 m² cm⁻³.

24. The method of any of claims 1-23, further comprising bioprinting, electrospinning, and/or melt electrowriting a third printing composition onto or into the non-filamentous scaffold.

25. The method of claim 24, wherein the third printing composition comprises a bioink composition.

26. The method of claim 24, wherein the bioink composition further comprises cells.

27. The method of claim 26, wherein the cells comprise pluripotent stem cells, multipotent stem cells, progenitor cells, terminally differentiated cells, endothelial cells, endothelial progenitor cells, immortalized cell lines, primary cells, or any combination thereof.

28. The method of any of claims 8-27, wherein the bioink composition comprises a polymer.

29. The method of claim 28, wherein the polymer comprises a hydrogel-forming agent.

30. The method of any of claims 28-29, wherein the polymer comprises a polysaccharide, such as alginate, hyaluronic acid, agarose, or any combination thereof.

31. The method of any of claims 28-30, wherein the polymer comprises a protein or peptide, such as gelatin, collagen, or any combination thereof.

32. The method of any of claims 28-31, wherein the polymer comprises a synthetic polymer, such as a polyester (e.g., poly(propylene fumarate) (PPF), polycaprolactone, poly(lactic-co-glycolic acid), polylactic acid, polyglycolic acid, or any combination thereof).

33. The method of any of claims 28-32, wherein the polymer is crosslinkable.

34. The method of any of claims 28-33, wherein the polymer is present in an amount of from 0.5% to 20% by weight, based on the total weight of the bioink composition.

35. The method of any of claims 28-34, wherein the bioink composition comprises a bioactive agent, such as a growth factor, growth inhibitor, cytokine, steroid, antibiotic, morphogen, or any combination thereof.

36. The method of claim 35, wherein the bioink composition comprises a polymer and wherein the bioactive agent is conjugated to the polymer.

37. The method of claim 35, wherein the bioink composition comprises a population of nanoparticles, a population of microparticles, or any combination thereof, and wherein the bioactive agent is conjugated to the particles.

38. The method of claim 35, wherein the bioink composition comprises a population of nanoparticles, a population of microparticles, or any combination thereof, and wherein the bioactive agent is encapsulated or dispersed in the particles.

39. The method of any of claims 10-38, wherein the fugitive ink composition comprises a polymer.

40. The method of claim 39, wherein the polymer comprises a poly(alkylene oxide) block copolymer, such as a polyoxyethylene-polyoxypropylene (PEO-PPO) block copolymers (e.g., a poloxamer).

41. The method of claim 39, wherein the polymer comprises hydroxyethyl cellulose (HEC).

42. The method of any of claims 39-41, wherein the polymer is present in an amount of from 0.5% to 20% by weight, based on the total weight of the fugitive ink composition.

43. The method of any of claims 1-42, further comprising using a multiplexer to select various chaotically printed microstructured precursors that are co-extruded to produce the non-filamentous microstructured precursor.

44. A microvascular appendage sheet made by the method of any of claims 1-43.
